(19) United States
(12) Patent Application Publication
(10) Pub. No.: US 2022/0380753 A1
(43) Pub. Date: Dec. 1, 2022

(54) EXPERIMENT AND MACHINE-LEARNING TECHNIQUES TO IDENTIFY AND GENERATE HIGH AFFINITY BINDERS

(71) Applicant: X Development LLC, Mountain View, CA (US)

(72) Inventors: Ivan Grubisic, Oakland, CA (US); Ray Nagatani, San Francisco, CA (US); Lance Co Ting Keh, La Crescenta, CA (US); Andrew Weitz, Los Altos, CA (US); Kenneth Jung, Mountain View, CA (US); Ryan Poplin, Fremont, CA (US)

(73) Assignee: X Development LLC, Mountain View, CA (US)

(21) Appl. No.: 17/333,272

(22) Filed: May 28, 2021

(51) Int. Cl. C12N 15/10; G16B 35/00; G16B 15/30

(52) U.S. Cl. CPC C12N 15/1048; G16B 35/00; G16B 15/30

(57) ABSTRACT

The present disclosure relates to in vitro experiments and in silico computation and machine-learning based techniques to iteratively improve a process for identifying binders that can bind any given molecular target. Particularly, aspects of the present disclosure are directed to obtaining sequence data for aptamers that bind to a target, where the sequence data has a first signal to noise ratio, generating, by a search process, a first set of aptamer sequences derived from the sequence data, obtaining subsequent sequence data for subsequent aptamers that bind to the target, where the subsequent aptamers includes aptamers synthesized from the first set of aptamer sequences, and the subsequent sequence data has a second signal to noise ratio greater than the first signal to noise ratio, generating, by a linear machine-learning model, a second set of aptamer sequences derived from the subsequent sequence data, and outputting the second set of aptamer sequences.

### FIELD

The present disclosure relates to development of aptamers, and in particular to in vitro experiments and in silico computation and machine-learning based techniques to iteratively improve a process for identifying binders that can bind any given molecular target.

### BACKGROUND

Aptamers are short sequences of single-stranded oligonucleotides (e.g., anything that is characterized as a nucleic acid, including xenobases). The sugar backbone of the single-stranded oligonucleotides functions as the acid and the A (adenine), T (thymine), C (cytosine), G (guanine) refers to the base. An aptamer can involve modifications to either the acid or the base. Aptamers have been shown to selectively bind to specific targets (e.g., proteins, protein complexes, peptides, carbohydrates, inorganic molecules, organic molecules such as metabolites, cells, etc.) with high binding affinity. Further, aptamers can be highly specific, in that a given aptamer may exhibit high binding affinity for one target but low binding affinity for many other targets. Thus, aptamers can be used to (for example) bind to disease-signature targets to facilitate a diagnostic process, bind to a treatment target to effectively deliver a treatment (e.g., a therapeutic or a cytotoxic agent linked to the aptamer), bind to target molecules within a mixture to facilitate purification, bind to a target to neutralize its biological effects, etc. However, the utility of an aptamer hinges on a degree to which it effectively binds to a target.

Frequently, an iterative experimental process (e.g., Systematic Evolution of Ligands by EXponential Enrichment (SELEX)) is used to identify aptamers that are selectively bound to target molecules with high affinity. In the iterative experimental process, a nucleic acid library of oligonucleotide strands (aptamers) is incubated with a target molecule. Then, the target-bound oligonucleotide strands are separated from the unbound strands and amplified via polymerase chain reaction (PCR) to seed a new pool of oligonucleotide strands. This selection process is continued for a number (e.g., 6-15) rounds with increasingly stringent conditions, which ensure that the oligonucleotide strands obtained have the highest affinity to the target molecule.

The nucleic acid library typically includes 10¹⁴-10¹⁵ random oligonucleotide strands (aptamers). However, there are approximately a septillion (10²⁴) different aptamers that could be considered. Exploring this full space of candidate aptamers is impractical. However, given that present-day experiments are now only a sliver of the full space, it is highly likely that optimal aptamer selection is not currently being achieved. This is particularly true when it is important to assess the degree to which aptamers bind with multiple different targets, as only a small portion of aptamers will have the desired combination of binding affinities across the targets. Accordingly, while substantive studies on aptamers have progressed since the introduction of the SELEX process, it would take an enormous amount of resources and time to experimentally evaluate a septillion (10²⁴) different aptamers every time a new target is proposed. In particular, there is a need for improving upon current experimental limitations with scalable machine-learning modeling techniques to identify aptamers and derivatives thereof that selectively bind to target molecules with high affinity.

### SUMMARY

In various embodiments, a method is provided that comprises: obtaining initial sequencing data for each unique aptamer of an initial aptamer library that binds to a target, where the initial sequence data has a first signal to noise ratio; generating, by a search process, a first set of aptamer sequences as an initial solution for a given problem, where the first set of aptamer sequences are derived from the initial sequencing data; obtaining subsequent sequencing data for each unique aptamer of a subsequent aptamer library that binds to the target, where the subsequent aptamer library comprises aptamers synthesized from the first set of aptamer sequences, and where the subsequent sequence data has a second signal to noise ratio that is greater than the first signal to noise ratio; generating, by a linear machine-learning model, a second set of aptamer sequences as a final solution for the given problem, where the second set of aptamer sequences are derived from the subsequent sequencing data; and outputting the second set of aptamer sequences.

In some embodiments, the search process comprises: (a) obtaining an initial population of aptamer sequences, where the initial population is a subset of sequences from the initial sequence data, sequences from a pool of sequences different from the sequences from the initial sequence data, or a combination thereof; (b) inputting the initial population into a nonlinear machine-learning model; (c) estimating, by the nonlinear machine-learning model, a fitness score of each aptamer sequence of the initial population, where the fitness scores is a measure of how well a given aptamer sequence performs as a solution with respect to the given problem; (d) selecting pairs of aptamer sequences from the initial population based on the fitness score for each aptamer sequence; (e) mating each pair of aptamer sequences by exchanging nucleotides between the pair of aptamer sequences up to a crossover point to generate offspring; (f) adding the offspring from each pair of aptamer sequences into a new population; (g) repeating steps (b)-(f) to create a sequence of new populations until a stopping criteria is met; and in response to meeting the stopping criteria, outputting a latest new population from step (f) as the first set of aptamer sequences.

In some embodiments, the estimating the fitness score of each aptamer sequence of the initial population, comprises generating, by the nonlinear machine-learning model, an uncertainty score for the fitness score of each aptamer sequence of the initial population; the uncertainty score is a quantification of uncertainty in a estimation of a fitness score by the nonlinear machine-learning model; and pairs of aptamer sequences from the initial population are selected based on the fitness score and uncertainty score for each aptamer sequence.

In some embodiments, the generating, by the linear machine-learning model, the second set of aptamer sequences, comprises: performing, using the subsequent sequencing data, a linear regression analysis to quantify a relationship between independent and dependent variables; determining a contribution of each independent to a value of a dependent value based on the relationship between the independent and the dependent variables; identifying the second set of aptamer sequences based on the contribution of each independent to the value of the dependent value; and outputting the second set of aptamer sequences.

In some embodiments, the nonlinear machine-learning model comprises greater than or equal to 10,000 parameters learned using: (i) a first set of training data comprising a subset of sequences from the initial sequence data, and (ii) a first objective function; the linear machine-learning model comprises less than 10,000 parameters learned using: (i) a second set of training data comprising a subset of sequences from the subsequent sequence data, and (ii) a second objective function; the second objective function is optimized, by linear programming, under linear equality and/or inequality constraint of a loss function; and regularized regression is applied to the second objective function by constraining at least one coefficient to zero.

In some embodiments, the method further comprises: synthesizing a final set of aptamers using the second set of aptamer sequences; validating, using a high-throughput or low-throughput affinity assay, one or more aptamers from the final set of aptamers capable of binding the target and solving the given problem; and synthesizing a biologic using the one or more aptamers validated as being capable of binding the target and solving the given problem.

In some embodiments, the method further comprises: receiving a query concerning potential therapeutic candidates that can bind the target and solve the given problem; acquiring the initial aptamer library as potentially satisfying the query; synthesizing a final set of aptamers using the second set of aptamer sequences; validating, using a high-throughput or low-throughput affinity assay, one or more aptamers from the final set of aptamers capable of binding the target and solving the given problem; and upon validating the one or more aptamers and in response to the query, providing aptamer sequences for the one or more aptamers as a result to the query.

In various embodiments, a method is provided that comprises: obtaining initial sequence data for each unique aptamer of an initial aptamer library that binds to a target; measuring a first signal to noise ratio within the initial sequence data; provisioning, based on the first signal to noise ratio, a first machine-learning system for generating a first set of aptamer sequences derived from the initial sequence data, where the provisioning comprises selecting or modifying one or more algorithms or models, modifying one or more model parameters of a preexisting algorithm or model, modifying one or more hyperparameters of a preexisting algorithm or model, augmenting the initial sequence data with additional data, selecting or modifying a training, testing, or validating approach for the one or more algorithms or the preexisting algorithm, modifying an objective or loss function of the one or more algorithms or the preexisting algorithm, or any combination thereof; generating, by the first machine-learning system, the first set of aptamer sequences as an initial solution for a given problem; obtaining subsequent sequence data for each unique aptamer of a subsequent aptamer library that binds to the target, where the subsequent aptamer library comprises aptamers synthesized from the first set of aptamer sequences; measuring a second signal to noise ratio within the subsequent sequence data; provisioning, based on the second signal to noise ratio, a second machine-learning system for generating a second set of aptamer sequences derived from the subsequent sequence data, where the provisioning comprises selecting or modifying one or more algorithms or models, modifying one or more model parameters of a preexisting algorithm or model, modifying one or more hyperparameters of a preexisting algorithm or model, augmenting the initial sequence data with additional data, selecting or modifying a training, testing, or validating approach for the one or more algorithms or the preexisting algorithm, modifying an objective or loss function of the one or more algorithms or the preexisting algorithm, or any combination thereof; generating, by the second machine-learning system, the second set of aptamer sequences as a final solution for the given problem; and outputting the second set of aptamer sequences.

In some embodiments, the initial aptamer library is determined, using a binding selection process, from a first Xeno nucleic acid (XNA) aptamer library synthesized from one or more single stranded DNA (deoxyribonucleic acid) or RNA (ribonucleic acid) libraries; the measuring the first signal to noise ratio comprises: (i) quantifying a number of unique aptamers in the initial aptamer library, quantifying a number of copies of each unique aptamer in the initial aptamer library, and determining a sequencing depth of the initial sequence data for each unique aptamer, and (ii) quantifying the first signal to noise ratio based on the quantification of the number of unique aptamers, the quantification of the copies of each unique aptamer, and the sequencing depth of the initial sequence data for each unique aptamer; the subsequent aptamer library is determined, using the binding selection process, from a second XNA aptamer library synthesized from the first set of aptamer sequences; and the measuring the second signal to noise ratio comprises: (i) quantifying a number of unique aptamers in the subsequent aptamer library, quantifying a number of copies of each unique aptamer in the subsequent aptamer library, and determining a sequencing depth of the subsequent sequence data for each unique aptamer, and (ii) quantifying the second signal to noise ratio based on the quantification of the number of unique aptamers, the quantification of the copies of each unique aptamer, and the sequencing depth of the subsequent sequence data for each unique aptamer.

In some embodiments, the one or more algorithms or models provisioned for the first machine-learning system comprise a first machine-learning model and a search algorithm; the first machine-learning model comprises model parameters learned using: (i) a first set of training data comprising a subset of sequences from the initial sequence data, and (ii) a first objective function; and the provisioning comprises selecting or modifying a first machine-learning algorithm or model and a search algorithm, modifying the model parameters of the first machine-learning algorithm or model, modifying one or more hyperparameters of the first machine-learning algorithm or model, augmenting the initial sequence data with additional data to generate the first set of training data, selecting or modifying a training, testing, or validating approach for the first machine-learning algorithm, modifying an objective or loss function of the first machine-learning algorithm, or any combination thereof.

In some embodiments, the generating the first set of aptamer sequences, comprises: (a) obtaining an initial population of aptamer sequences, where the initial population is a subset of sequences from the initial sequence data, sequences from a pool of sequences different from the sequences from the initial sequence data, or a combination thereof; (b) inputting the initial population into the first machine-learning model; (c) estimating, by the first machine-learning model, a fitness score of each aptamer sequence of the initial population, where the fitness scores is a measure of how well a given aptamer sequence performs as a solution with respect to the given problem; (d) selecting, by the search algorithm, pairs of aptamer sequences from the initial population based on the fitness score for each aptamer sequence; (e) mating, by the search algorithm, each pair of aptamer sequences by exchanging nucleotides between the pair of aptamer sequences up to a crossover point to generate offspring; (f) adding the offspring from each pair of aptamer sequences into a new population; (g) repeating steps (b)-(f) to create a sequence of new populations until a stopping criteria is met; and in response to meeting the stopping criteria, outputting a latest new population from step (f) as the first set of aptamer sequences.

In some embodiments, the one or more algorithms or models provisioned for the second machine-learning system comprise a second machine-learning model; the second machine-learning model comprises model parameters learned using: (i) a second set of training data comprising a subset of sequences from the subsequent sequence data, and (ii) a second objective function; and the provisioning comprises selecting or modifying a second machine-learning algorithm or model, modifying the model parameters of the second machine-learning algorithm or model, modifying one or more hyperparameters of the second machine-learning algorithm or model, augmenting the subsequent sequence data with additional data to generate the second set of training data, selecting or modifying a training, testing, or validating approach for the second machine-learning algorithm, modifying an objective or loss function of the second machine-learning algorithm, or any combination thereof.

In some embodiments, the generating the second set of aptamer sequences comprises: performing, by the second machine-learning model using the subsequent sequence data, a regression analysis to quantify a relationship between independent and dependent variables; determining, by the second machine-learning model, a contribution of each independent to a value of a dependent value based on the relationship between the independent and the dependent variables; identifying, by the second machine-learning model, the second set of aptamer sequences based on the contribution of each independent to the value of the dependent value; and outputting, by the second machine-learning model, the second set of aptamer sequences.

In some embodiments, the method further comprises: synthesizing a final set of aptamers using the second set of aptamer sequences; validating, using a high-throughput or low-throughput affinity assay, one or more aptamers from the final set of aptamers capable of binding the target and solving the given problem; and synthesizing a biologic using the one or more aptamers validated as being capable of binding the target and solving the given problem.

In some embodiments, the method further comprises: receiving a query concerning potential therapeutic candidates that can bind the target and solve the given problem; acquiring the initial aptamer library as potentially satisfying the query; synthesizing a final set of aptamers using the second set of aptamer sequences; validating, using a high-throughput or low-throughput affinity assay, one or more aptamers from the final set of aptamers capable of binding the target and solving the given problem; and upon validating the one or more aptamers and in response to the query, providing aptamer sequences for the one or more aptamers as a result to the query.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure will be better understood in view of the following non-limiting figures, in which:

FIG. 1 shows a block diagram of a pipeline for strategically identifying and generating high affinity binders of molecular targets according to various embodiments;

FIG. 2 shows a machine-learning modeling system for developing aptamers in accordance with various embodiments;

FIG. 3 shows a block diagram of a aptamer development platform according to various embodiments;

FIG. 4 shows an exemplary flow for aptamer development in accordance with various embodiments;

FIG. 5 shows an exemplary flow for aptamer development using a predefined pipeline in accordance with various embodiments;

FIG. 6 shows an exemplary flow for aptamer development using a dynamic pipeline in accordance with various embodiments; and

FIG. 7 shows an exemplary computing device in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. INTRODUCTION

Identification of high affinity and high specificity binders (e.g., monoclonal antibodies, nucleic acid aptamers, and the like) of molecular targets (e.g., VEGF, HER2) has dramatically transformed treatment of many types of diseases (e.g., oncology, infectious disease, immune/inflammation, etc.). However, given the large search space of potential sequences (e.g., 10²⁴ potential sequences for the average aptamer or monoclonal antibody CDR-H3 binding loop) and the comparatively low-throughput of methodologies to assess the binding affinity of candidates (e.g., dozens to thousands per week) it is highly likely that optimal binder selection is not currently being achieved. While selection based approaches (e.g., phage display, SELEX, and the like) can potentially identify binders, among libraries of millions to trillions of candidates, there are several weaknesses with these approaches: (i) output is binary—it is challenging to know whether relatively strong binders in the library are actually strong binders; (ii) data is noisy—binding is dependent on every candidate encountering available target with the same relative frequency and variance from this can lead to many false negatives and some false positives; and (iii) capacity is much smaller than the total search space—phage display (max candidates ˜10⁹) and SELEX (max candidates ˜10¹⁴) search spaces much smaller than the total possible search space (additionally, it is generally difficult (or expensive) to characterize the portions of the total sequence space that are searched).

To address these challenges, efforts have been made to apply computational and machine learning techniques in an “experiment in the loop” process to reduce the search space and design better binders. For example, the following computational and machine learning techniques have been attempted to increase discovery of viable high affinity/high specificity binders of molecular targets: (i) identification of libraries more likely to bind via prediction from physics based models, (ii) input of selection data and design/identify more likely binders (for monoclonal antibodies and nucleic acid aptamers), and (iii) address other factors beyond affinity that affect commercialization and therapeutic potential. To date however, these computational and machine learning techniques have had limited success in designing markedly different sequences with better properties, let alone with sufficient predictive power to align on a small set of sequences appropriate for low-throughput characterization. Particularly, the techniques in the second category, often struggle to input sufficient data to identify or design candidates that are markedly different from the training sequences used to train the computation and machine learning models.

To address these limitations and others, an aptamer development system is disclosed herein that derives in silico aptamers sequences from in vitro aptamer sequences found experimentally to bind to a target. For instance in an exemplary embodiment, a predefined developmental process may comprise: obtaining initial sequencing data for each unique aptamer of an initial aptamer library that binds to a target, where the initial sequence data has a first signal to noise ratio; generating, by a search process, a first set of aptamer sequences as an initial solution for a given problem, where the first set of aptamer sequences are derived from the initial sequencing data; obtaining subsequent sequencing data for each unique aptamer of a subsequent aptamer library that binds to the target, where the subsequent aptamer library comprises aptamers synthesized from the first set of aptamer sequences, and where the subsequent sequence data has a second signal to noise ratio that is greater than the first signal to noise ratio; generating, by a linear machine-learning model, a second set of aptamer sequences as a final solution for the given problem, where the second set of aptamer sequences are derived from the subsequent sequencing data; and outputting the second set of aptamer sequences. The signal to noise ratio within the various in vitro aptamer sequences is used as a metric to drive decisions on the types of machine-learning techniques provisioned within the aptamer development system to derive the in silico aptamers sequences. Advantageously, the less noise in a data set of sequences the more confidence there is to provision components of the aptamer development system to go from identifying or designing sequences in-sample domain (stay near training data) to out-of-sample domain (further away from training data).

In an exemplary alternative embodiment, a dynamic developmental process may comprise: obtaining initial sequence data for each unique aptamer of an initial aptamer library that binds to a target; measuring a first signal to noise ratio within the initial sequence data; provisioning, based on the first signal to noise ratio, a first machine-learning system for generating a first set of aptamer sequences derived from the initial sequence data, where the provisioning comprises selecting or modifying one or more algorithms or models, modifying one or more model parameters of a preexisting algorithm or model, modifying one or more hyperparameters of a preexisting algorithm or model, augmenting the initial sequence data with additional data, selecting or modifying a training, testing, or validating approach for the one or more algorithms or the preexisting algorithm, modifying an objective or loss function of the one or more algorithms or the preexisting algorithm, or any combination thereof; generating, by the first machine-learning system, the first set of aptamer sequences as an initial solution for a given problem; obtaining subsequent sequence data for each unique aptamer of a subsequent aptamer library that binds to the target, where the subsequent aptamer library comprises aptamers synthesized from the first set of aptamer sequences; measuring a second signal to noise ratio within the subsequent sequence data; provisioning, based on the second signal to noise ratio, a second machine-learning system for generating a second set of aptamer sequences derived from the subsequent sequence data, where the provisioning comprises selecting or modifying one or more algorithms or models, modifying one or more model parameters of a preexisting algorithm or model, modifying one or more hyperparameters of a preexisting algorithm or model, augmenting the initial sequence data with additional data, selecting or modifying a training, testing, or validating approach for the one or more algorithms or the preexisting algorithm, modifying an objective or loss function of the one or more algorithms or the preexisting algorithm, or any combination thereof; generating, by the second machine-learning system, the second set of aptamer sequences as a final solution for the given problem; and outputting the second set of aptamer sequences. The signal to noise ratio within the various in vitro aptamer sequences is again used as a metric to drive decisions on the types of machine-learning techniques provisioned within the aptamer development system to derive the in silico aptamers sequences. Advantageously, in this instance the signal to noise ratio is measured after each experiment and the machine-learning system(s) are provisioned dynamically to best address the noise in a present data set of in vitro aptamer sequences.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of what is specified, where the percentage includes 0.1, 1, 5, and 10 percent.

As used herein, when an action is “based on” something, this means the action is based at least in part on at least a part of the something.

It will be appreciated that techniques disclosed herein can be applied to assess other biological material (e.g., other binders such as monoclonal antibodies) rather than aptamers. For example, alternatively or additionally, the techniques described herein may be used to assess the interaction between any type of biologic material (e.g., a whole or part of an organism such as E. coli, or a biologic product that is produced from living organisms, contain components of living organisms, or derived from human, animal, or microorganisms by using biotechnology) and a target, and derive another type of biologic material therefrom based on the assessment.

### II. PIPELINE TO IDENTIFY AND GENERATE HIGH AFFINITY BINDERS OF MOLECULAR TARGETS

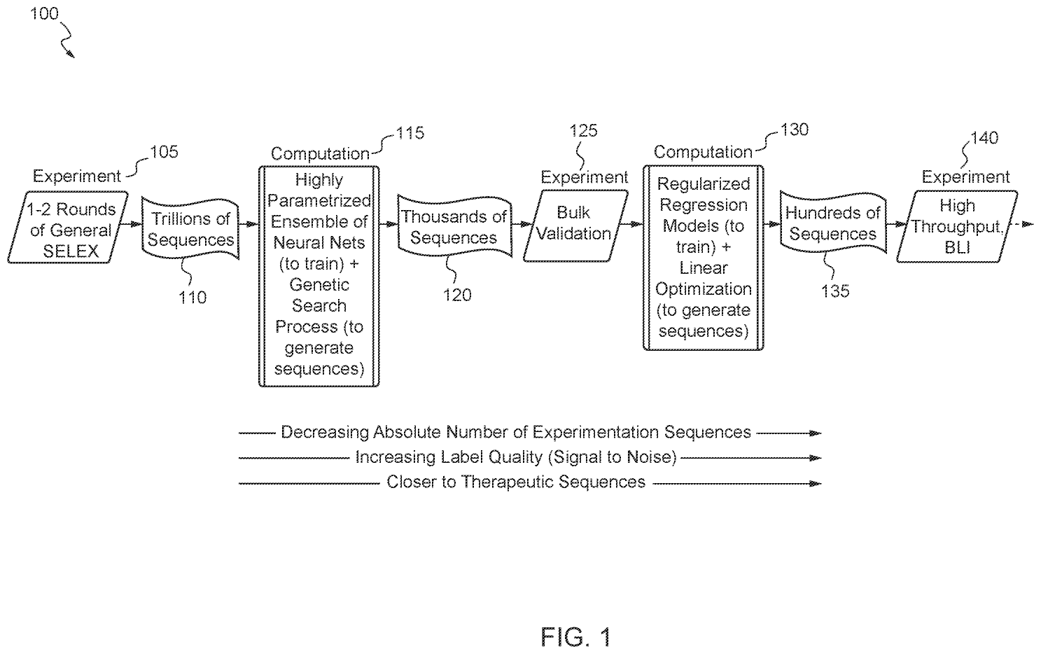

FIG. 1 shows a block diagram of a pipeline 100 for strategically identifying and generating high affinity binders of molecular targets. As used herein, the term “binding affinity” means the free energy differences between native binding and unbound states, which measures the stability of native binding states (e.g., a measure of the strength of attraction between an aptamer and a target). As used herein, a “high binding affinity” is a result from stronger intermolecular forces between an aptamer and a target leading to a longer residence time at the binding site (higher “on” rate, lower “off” rate). The factors that lead to high affinity binding include a good fit between surface of the molecules in their ground state and charge complementary (i.e., stronger intermolecular forces between the aptamer and the target). These same factors generally also provide a high binding specificity for the targets, which can be used to simplify screening approaches aimed at developing strong therapeutic candidates that can bind the given molecular target. As used herein, the term “binding specificity” means the affinity of binding to one target relative to the other targets. As used herein, the term “high binding specificity” means the affinity of binding to one target is stronger relative to the other targets. Various aspects described herein design and validate aptamers as strong therapeutic candidates that can bind the given molecular target based on binding affinity. However, it should be understood that design and validation of aptamers could involve the assessment of binding affinity and/or binding specificity.

In various embodiments, the pipeline 100 implements in vitro experiments and in silico computation and machine-learning based techniques to iteratively improve a process for identifying binders that can bind any given molecular target. At block 105, in vitro binding selections (e.g., phages display or SELEX) are performed where a given molecular target (e.g., a protein of interest) is exposed to tens of trillions of different potential binders (e.g., a library of 10¹⁴-10¹⁵ nucleic acid aptamers), a separation protocol is used to remove non-binding aptamers (e.g., flow-through), and the binding aptamers are eluted from the given target. The binding aptamers and the non-binding aptamers are sequenced to identify what aptamers do and do not bind the given target. This binding selection process may be repeated for any number of cycles (e.g., 1 to 3 cycles) to reduce the absolute count of potential binders from tens of trillions of different potential binders down to millions or trillions of binders 110 identified to have some level of binding (specific and non-specific) for the given target.

At block 110, the sequences of binding aptamers (and optionally non-binding aptamers) obtained from block 105 are used to train a highly parameterized machine-learning algorithm (i.e., a parameter count of greater than or equal to 10,000, 30,000, 50,000, or 75,000) and learn a fitness function capable of ranking the fitness (quality) of sequences of aptamers based on a problem being solved (e.g., binding to target with a high-affinity). Machine-learning algorithms are procedures that are implemented in code and are run on data to generate machine-learning models. The machine-learning models represent what was learned by the machine-learning algorithms during training. In other words, the machine-learning models are the data structures that are saved after running machine-learning algorithms on training data and represents the rules, variables, and any other algorithm-specific data structures required to make predictions. The use of a large data set with diverse sequences of binding aptamers (e.g., millions or trillions of binders) in the training allows for the algorithm to learn all of the parameters required for estimating the fitness of aptamer candidates for a given problem. Otherwise, the problem of having a large number parameters and dimensions yet small data sets results in overfitting, which means the learned function is too closely fit to a limited set of data points and works only for the data set the algorithm was trained with, rendering the learned parameters pointless. The model trained on the large data set from block 105 can then take as input sequences not necessarily discovered in the in vitro binding selections and estimate a fitness for those input sequences to solve the given problem. Thus, artificially increasing the search space for aptamers that can bind the target and solve the given problem from the 10¹⁴-10¹⁵ nucleic acid aptamers investigated in the in vitro experimentation stage to at least 10²⁴ nucleic acid aptamers and beyond depending on algorithm complexity and computational power required.

Nonetheless, there are challenges associated with estimating the fitness of additional or alternative sequences of aptamers using a highly parameterized machine-learning algorithm. During learning, the outputs of the algorithm may come to approximate target values given the inputs in the training set. This ability is useful in itself, but the purpose of using the highly parameterized machine-learning algorithm is to generalize, i.e., to have the outputs of the algorithm approximate target values given inputs that are not in the training set. Good generalization allows for the trained model to be able to identify or design aptamer candidates that are markedly different from the training sequences used to train the algorithm. Typically good generalization requires: (i) the inputs to the algorithm contain sufficient information pertaining to the target, so that there exists a mathematical function relating correct outputs to inputs with a desired degree of accuracy, (ii) the function being learned (that relates inputs to correct outputs) is, in some sense, smooth (a small change in the inputs should, most of the time, produce a small change in the outputs), (iii) the training set is sufficiently large and representative of a subset of the set of all cases that a user wants to generalize to, and (iv) there is limited noise in the inputs to the algorithm.

The sequences of binding aptamers (and optionally non-binding aptamers) obtained from block 105 are going to have a low signal to noise ratio (and low label quality) because of the large amount of noise (sequences of aptamers with non-specific binding or low affinity binding to the given target) in the sequences. Essentially, the signal to noise ratio is a fraction of tested aptamers that have the desired binding characteristics when assayed with high/low throughput characterization or validation. Typically, machine learning algorithms model two different parts of the training data—the underlying generalizable truth (the signal), and the randomness specific to that dataset (the noise). Fitting both of those parts can increase the training set accuracy, but fitting the signal also increases test set accuracy or generalization (and real-world performance) while fitting the noise decreases both the test set accuracy and real-world performance (causes overfitting). Thus, conventional regularization techniques such as L1 (lasso regression), L2 (ridge regression), dropout, and the like may be implemented in the training to make it harder for the algorithm to fit the noise, and so more likely for the algorithm to fit the signal and generalize more accurately.

However, conventional regularization techniques can lead to dimensionality reduction, which means the machine-learning model is built using a lower dimensional dataset (e.g., less parameters). This can lead to a high bias error in the outputs (known as underfitting). In order to overcome these challenges and others, aspects of the present disclosure are directed to using a combination of in silico computational and machine-learning based techniques (e.g., ensemble of neural nets, genetic search processes, regularized regression models, linear optimization, and the like) in combination with various in vitro experimentation techniques (e.g., binding selections, SELEX, and the like) to identify or design markedly different sequences with better properties, while maintaining sufficient predictive power to align on a small set of sequences (e.g., tens to hundreds) appropriate for low-throughput characterization or validation. In some instances, the various techniques are implemented in the pipeline 100 via a predefined architecture (e.g., the exemplary architecture shown in FIG. 1 and described herein) to decrease the absolute number of sequences being used as input for each stage while passively increasing the signal to noise ratio (e.g., decreasing the noise) and label quality, and to ultimately predict the highest quality binders (e.g., highest-affinity) for any given molecular target.

In other instances, the techniques are implemented in the pipeline 100 via a dynamic architecture to decrease the absolute number of sequences being used as input for each stage while actively increasing the signal to noise ratio (decreasing the noise) and label quality, and to ultimately predict the highest quality binders for any given molecular target. The active increase in the signal to noise ratio and label quality is implemented by: (i) measuring the amount of noise in the training data set at each stage, and (ii) provisioning components of the pipeline 100 in various stages to dynamically change the architecture for optimally addressing the measured amount of noise and label quality of the input sequences. As used herein, the term “provisioning” means the selection, deployment, and run-time management of software (e.g., algorithms and models) and hardware resources (e.g., CPU, storage, and network) for ensuring performance for aptamer development applications. The provisioning includes modifying the algorithms or models being used at various stages (e.g., implementing a neural network versus implementing a regression model), modifying one or more model parameters (e.g., adding or removing weights from various connections), modifying one or more hyperparameters (e.g., adding or removing a hidden layer), augmenting the input sequences or training set of data (e.g., artificially manipulating the sequences to increase the signal or reduce the noise from the training set of data), modifying the training/testing/validating approach (e.g., using an ensemble based learning approach versus a transfer learning approach), modifying the objective or loss function for a given algorithm (e.g., using mean squared error loss versus mean squared logarithmic error loss), or any combination thereof.

With reference back to FIG. 1, in some instances, the highly parameterized machine-learning algorithm (i.e., a parameter count of greater than or equal to 10,000, 30,000, 50,000, or 75,000) used in block 115 is a series of algorithms such as a neural network. A series of algorithms offers increased flexibility and can scale in proportion to the amount of training data available. A downside of this flexibility is that the algorithms learn via a stochastic training algorithm which means that the algorithms are sensitive to both the specific training data set (presumed to be a random sample from some fixed distribution) and also the initial conditions, etc., of the training run (e.g., seeds for pseudo-random number generators). Additionally, there is also randomness that is hard to control for even if random seeds are set because modern GPUs (presumably TPUs) are not guaranteed deterministic. This means that the algorithms are subject to overfitting and can have high variance when it comes to making a final prediction (e.g., prediction of a fitness score for additional or alternative sequences of aptamers). In order to overcome this variance, in some instances, the highly parameterized machine-learning algorithm is provisioned as a series of multiple neural networks trained using an ensemble based approach to combine the predictions from the multiple neural networks. Combining the predictions from multiple neural networks counters the variance of a single trained neural network model and can reduce generalization error (also known as the out-of-sample error, which is a measure of how accurately an algorithm is able to predict outcome values for previously unseen data). For example, generalization error is typically decomposed into bias and variance; bias is (roughly) reduced by more expressive models (e.g., neural nets with many more parameters) but increasing the flexibility of models can lead to overfitting. Variance is (roughly) reduced by ensembles or larger datasets. Thus, for instance, random forests are ensembles of very flexible models (decisions trees)—the low bias of the component models usually lead to high variance solutions, so this can be counteracted by using an ensemble of trees, each fit to a random subset (optionally along with other techniques) of the data The results of the ensemble of neural networks are predictions that are less sensitive to the specifics of the training data, choice of training scheme, and the randomness inherent in a single training run.

The trained highly parameterized machine-learning model (e.g., an ensemble of neural networks) may then be used in a search process to predict fitness scores and identify thousands of other sequences of aptamers 120 that can potentially bind the given target. In some instances, the search process is a genetic search process that uses a genetic algorithm, which mimics the process of natural selection, where the fittest individuals (e.g., aptamers with a potential for binding a given target) are selected for reproduction in order to produce offspring of the next generation (e.g., aptamers with the greatest potential for binding the given target). If parents have better fitness, their offspring will be better than parents and have a better chance at surviving. This process keeps on iterating and at the end, a generation with the fittest individuals (e.g., thousands of sequences of aptamers 120 with the best potential for binding the given target) will be found. In certain instances, the genetic algorithm is constrained to a limited number of nucleotide edits away from the training dataset knowing that variance of empirical labels relative to highly parameterized machine-learning model predictions increases drastically.

At block 125, identified or designed sequences of aptamers 120 may be used to synthesize aptamers, which are used for subsequent binding selections. For example, subsequent in vitro binding selections (e.g., phages display or SELEX) may be performed where the given molecular target is exposed to the synthesized aptamers, a separation protocol is used to remove non-binding aptamers (e.g., flow-through), and the binding aptamers are eluted from the given target. The binding and non-binding aptamers are sequenced to identify what aptamers do and do not bind the given target. This binding selection process may be repeated for any number of cycles (e.g., 1 to 3 cycles) to validate which of the identified/designed aptamers actually bind the given target. In some instances, the subsequent binding selections are performed using Unique Molecular Identifiers (UMI) to enable accurate counting of copies of a given candidate sequence in elution or flow-through. Because the sequence diversity is reduced at this stage, there can be more copies of each aptamer to interact with the given target and improve the signal to noise ratio (and label quality).

At block 130, the sequences of binding aptamers (and optionally non-binding aptamers) obtained from block 125 are used to train a linear algorithm to identify hundreds of additional or alternative sequences of aptamers 135 that can potentially bind the given target. In some instances, the linear algorithm is a multiple regression algorithm learned using regularization techniques (i.e., fitting a model with more than one independent variable (covariates or predictors or features—all the same thing)) to obtain a multiple regularized regression model. While the linear algorithms are less expressive than highly parametrized algorithms, the improved signal to noise at this stage allows the linear algorithms to still capture signal while being better at generalizing. Optimization techniques such as linear optimization may be is used at this stage to identify the hundreds of additional or alternative sequences of aptamers 135 with differing relative fitness scores (and therefore affinity). Linear optimization (also called linear programming) is a computational method to achieve the best outcome (such as highest binding affinity for a given target) in a model whose requirements are represented by linear relationships (e.g., a regression model). More specifically, the linear optimization improves the linear objective function, subject to linear equality and linear inequality constraints to output the hundreds of additional or alternative sequences of aptamers 135 with differing relative fitness scores (including those with a highest binding affinity). Unlike the highly parameterized machine-learning model and searching process used in block 115, there is greater confidence in deviating away from training data in the process of linear optimization due to better generalization by the regression models. Consequently, the linear optimization may not be constrained to a limited number of nucleotide edits away from the training dataset.

At block 140, identified or designed sequences of aptamers 135 may be used to design aptamers, which are subsequently characterized or validated in either high throughput binding selections (e.g., SELEX) or low-throughput affinity assays (e.g., biolayer interferometry (BLI)) for binding the given target. The processes in blocks 105-140 may be performed once or repeated in part or in their entirety any number of times to decrease the absolute number of sequences and increase the signal to noise ratio, which ultimately results in a set of strong therapeutic candidates that can bind the given molecular target (e.g., bind targets of interest in a inhibitory/activating fashion or to deliver a drug/therapeutic to a target such as a T-Cell). It will be appreciated that although FIG. 1 and the description herein, describe going from trillions of sequences to thousands of sequences to hundreds of sequences, these numbers are merely provided for illustrative purposes. In general, it should be understood that pipeline 100 is provisioned to start with a large data set (a large absolute number of experimentation sequences which could be, for example, septillions, trillions, billions, or millions) for training a highly-parametrized algorithm and eventually narrows down the absolute number of experimentation sequences to a more manageable number eventually aligning on a small data set (a small absolute number of experimentation sequences which could be, for example, hundreds, tens, or less) for low-throughput characterization and validation as potential therapeutic candidates.

### III. MODELING SYSTEMS TO IDENTIFY/DESIGN SEQUENCES FOR BINDERS

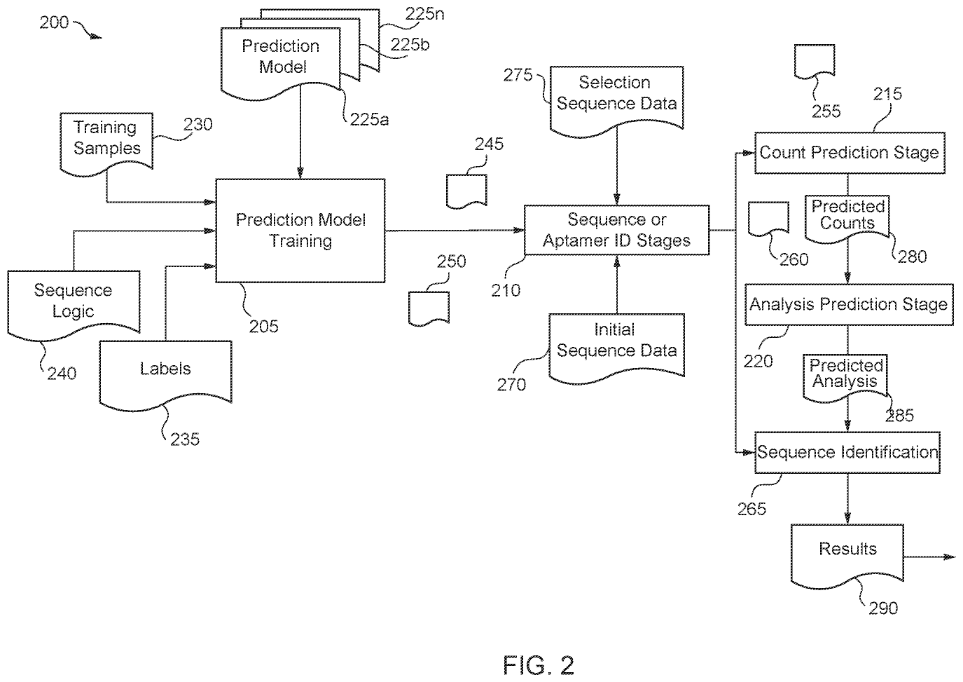

FIG. 2 shows a block diagram illustrating aspects of a machine-learning modeling system 200 for identifying or designing high affinity binders (e.g., aptamers, peptides, proteins, or peptidomimetics that answer a query posed by a user) of molecular targets. As shown in FIG. 2, the predictions performed by the machine-learning modeling system 200 in this example include several stages: a prediction model training stage 205, one or more sequence or aptamer identification stages 210, an optional count prediction stage 215, and an optional analysis prediction stage 220. The prediction model training stage 205 builds and trains one or more models 225a-225n (‘n’ represents any natural number) to be used by the other stages (which may be referred to herein individually as a model 225 or collectively as the models 225). For example, the models 225 can include one or more different type of models for generating sequences of aptamers not experimentally determined by a selection process but identified or designed based on aptamers experimentally determined by a selection process. The models 225 may be used in the pipeline 100 described with respect FIG. 1 for identifying or designing high affinity binders for a given target. The models 225 can also include a model for predicting binding counts for the predicted sequences for derived aptamers. The models 225 can also include a model for predicting analytics such as binding affinity for the predicted sequences for derived aptamers. Still other types of prediction models may be implemented in other examples according to this disclosure.

A model 225 can be a machine-learning model, such as a neural network, a convolutional neural network (“CNN”), e.g. an inception neural network, a residual neural network (“Resnet”) or NASNET provided by GOOGLE LLC from MOUNTAIN VIEW, CALIFORNIA, or a recurrent neural network, e.g., long short-term memory (“LSTM”) models or gated recurrent units (“GRUs”) models. A model 225 can also be any other suitable machine-learning model trained to predict predicted sequences for derived aptamers, sequence counts or analytics for aptamer sequences, such as a support vector machine, decision tree, a three-dimensional CNN (“3DCNN”), regression model, linear regression model, ridge regression model, logistic regression model, a dynamic time warping (“DTW”) technique, a hidden Markov model (“HMM”), etc., or combinations of one or more of such techniques—e.g., CNN-HMM or MCNN (Multi-Scale Convolutional Neural Network). The machine-learning modeling system 200 may employ one or more of same type of model or different types of models for aptamer sequence prediction, aptamer count prediction, and/or analysis prediction.

To train the various models 225 in this example, training samples 230 for each model 225 are obtained or generated. The training samples 230 for a specific model 225 can include the sequence data as described with respect to FIG. 1 and optional labels 235 corresponding to the sequence data. For example, for a model 225 to be utilized to identify or design an aptamer sequence, the input can be the aptamer sequence itself or features extracted from the sequence data associated with the aptamer sequence and optional labels 235 can include calculated fitness scores for the aptamer sequences (a measure of how well each aptamer sequences solves a given problem). Similarly, for a model 225 to be utilized to predict a count or binding affinity for an aptamer sequence, the input can include the sequence and count features extracted from the initial sequence data and/or the sequence data associated with the sequence, and the optional labels 235 can include features indicating parameters for the count or binding affinity or a vector indicating probabilities for the count or binding affinity of the sequence data.

In some instances, the training process includes iterative operations to find a set of parameters for the model 225 that maximizes or minimizes an objective function (e.g., regression or classification loss) for the models 225. Each iteration can involve finding a set of parameters for the model 225 so that the value of the objective function using the set of parameters is smaller or greater than the value of the objective function using another set of parameters in a previous iteration. The objective function can be constructed to measure the difference between the outputs predicted using the models 225 and the optional labels 235 contained in the training samples 230. Once the set of parameters are identified, the model 225 has been trained and can be tested, validated, and/or utilized for prediction as designed.

In addition to the training samples 230, other auxiliary information can also be employed to refine the training process of the models 225. For example, sequence logic 240 can be incorporated into the prediction model training stage 205 to ensure that the sequences or aptamers, counts, and analysis predicted by a model 225 do not violate the sequence logic 240. For example, binding affinity (the strength of the binding interaction between an aptamer and a target) is a characteristic that can drive aptamers to be present in greater numbers in a pool of aptamer-target complexes after a cycle of selection process. This relationship can be expressed in the sequence logic 240 such that as the binding affinity variable increases the predictive count increases (to represent this characteristic), as the binding affinity variable decreases the predictive count decreases. Moreover, an aptamer sequence generally has inherent logic among the different nucleotides. For example, GC content for an aptamer is typically not greater than 60%. This inherent logical relationship between GC content and aptamer sequences can be exploited to facilitate the aptamer sequence prediction.

According to some aspects of the disclosure presented herein, the logical relationship between the binding affinity and count can be formulated as one or more constraints to the optimization problem for training the models 225. A training loss function that penalizes the violation of the constraints can be built so that the training can take into account the binding affinity and count constraints. Alternatively, or additionally, structures, such as a directed graph, that describe the current features and the temporal dependencies of the prediction output can be used to adjust or refine the features and predictions of the models 225. In an example implementation, features may be extracted from the initial sequence data and combined with features from the selection sequence data as indicated in the directed graph. Features generated in this way can inherently incorporate the temporal, and thus the logical, relationship between the initial library and subsequent pools of aptamer sequences after cycles of the selection process. Accordingly, the models 225 trained using these features can capture the logical relationships between sequence characteristics, selection cycles, aptamer sequences, and nucleotides.

Although the training mechanisms described herein mainly focus on training a model 225, these training mechanisms can also be utilized to fine tune existing models 225 trained from other datasets. For example, in some cases, a model 225 might have been pre-trained using pre-existing aptamer sequence libraries. In those cases, the models 225 can be retrained using the training samples 230 containing initial sequence data, experimentally derived selection sequence data, and other auxiliary information as discussed herein.

The prediction model training stage 205 outputs trained models 225 including trained nonlinear or highly parametrized models 245, trained linear models or models with minimal parameters 250, optionally trained count prediction models 255, and optionally trained analysis prediction models 260. The trained nonlinear or highly parametrized models 245 and trained linear models or models with minimal parameters 250 may be used in the sequence identification stages 210 to identify or design sequences 265 based on a subset or all of the initial sequence data 270 (e.g., random sequence data), the selection sequence data 275 identified during the experimental selection process (e.g., blocks 105-140 described with respect to FIG. 1), or a combination thereof. The trained count prediction models 255 may be used in the count prediction stage 215 to generate count predictions 280 for the identified sequences based on the initial sequence data 270 and/or the selection sequence data 275 identified during the experimental selection process (e.g., blocks 105, 125, and 140 described with respect to FIG. 1). The trained analysis prediction models 260 may be used in the analysis prediction stage 220 to generate analysis predictions 285 (e.g., a binary classifier such as binds to target or does not bind to target) for the identified sequences based on the initial sequence data 270 and/or the selection sequence data 275 identified during the experimental selection process (e.g., blocks 105, 125, and 140 described with respect to FIG. 1). In some instances, the identified or designed sequences 265, count predictions 280, analysis predictions 285, or any combination thereof may be provided as results 290 to a query posed by a user. For example, in response to a query for top hundred aptamers that bind a given target, the results 290 may include the identity of sequences for a hundred aptamers with the highest count or binding affinity for the given target. As described with respect to FIG. 1, the results 290 may then be used to synthesize the aptamers to be used in low-throughput assays for characterizing or validating the results 290 as potential therapeutic candidates.

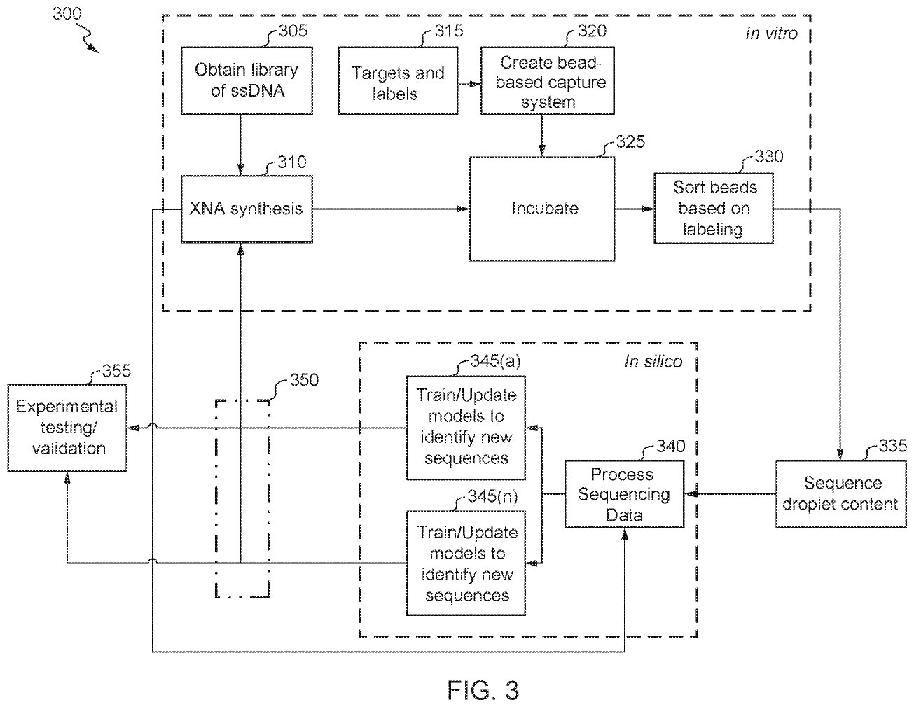

FIG. 3 shows a block diagram of an aptamer development platform 300 for strategically identifying and generating high affinity binders of molecular targets. In various embodiments, the aptamer development platform 300 implements in vitro experiments and in silico computation and machine-learning based techniques to iteratively improve a process for identifying binders that can bind any given molecular target. The various components of the aptamer development platform 300 are executed in accordance with the pipeline developed for identifying and generating high affinity binders of molecular targets (as described with respect to FIG. 1). The in silico computation and machine-learning based techniques are trained and deployed as at least part of a machine-learning modeling system (as described with respect to FIG. 2).

In various embodiments, the aptamer development platform 300 implements screening-based techniques for aptamer discovery where each candidate aptamer sequence in a library is assessed based on the query (e.g., binding affinity with one or more targets or functionally capable of inhibiting one or more targets) in a high throughput binding selection process. As described herein, the aptamer development platform 300 implements machine learning based techniques for enhanced aptamer discovery where candidate aptamer sequences in a library that satisfy the query are used to train one or more machine-learning models to identify additional or alternative candidate aptamer sequences that potentially satisfy the query. The aptamer development platform 300 further implements screening-based techniques for aptamer validation to validate or confirm that the identified aptamer candidate sequences do satisfy the query (e.g., bind or inhibit the one or more targets) in a high throughput or low throughput manner. As should be understood, these techniques from screening through identification to validation can be repeated in one or more closed loop processes sequentially or in parallel to ultimately assess any number of queries.

The aptamer development platform 300 includes obtaining one or more single stranded DNA (deoxyribonucleic acid) or RNA (ribonucleic acid) (ssDNA [single-stranded DNA] or ssRNA [single-stranded RNA]) libraries at block 305. The one or more sssDNA or ssRNA libraries may be obtained from a third party (e.g., an outside vendor) or may be synthesized in-house, and each of the one or more libraries typically contains up to 10¹⁷ different unique sequences. At block 310, the ssDNA or ssRNA of the one or more libraries are transcribed to synthesize a Xeno nucleic acid (XNA) aptamer library. XNA aptamer sequences (e.g., threose nucleic acids [TNA], 1,5-anhydrohexitol nucleic acid [HNA], cyclohexene nucleic acid [CeNA], glycol nucleic acid [GNA], locked nucleic acid [LNA], peptide nucleic acid [PNA], FANA [fluoro arabino nucleic acid]) are synthetic nucleic acid analogues that have a different sugar backbone than the natural nucleic acids DNA and RNA. XNA may be selected for the aptamer sequences as these polymers are not readily recognized and degraded by nucleases, and thus are well-suited for in vivo applications. XNA aptamer sequences may be synthesized in vitro through enzymatic or chemical synthesis. For example, an XNA library of aptamers may be generated by primer extension of some or all of the oligonucleotide strands in a ssDNA library, flanking the aptamer sequences with fixed primer annealing sites for enzymatic amplification, and subsequent PCR amplification to create an XNA aptamer library that includes 10¹²-10¹⁷ aptamer sequences.

In some instances, the XNA aptamer library may be processed for application in downstream machine-learning processes. In certain instances, the aptamer sequences are processed for use as training data, test data, or validation data in one or more machine-learning models. In other instances, the aptamer sequences are processed for use as actual experimental data in one or more trained machine-learning models. In either instance, the aptamer sequences may be processed to generate initial sequence data comprising a representation of the sequence of each aptamer and optionally a count metric. The representation of the sequence can include one-hot encoding of each nucleotide in the sequence that maintains information about the order of the nucleotides in the aptamer. The representation of the sequence can additionally or alternatively include a string of category identifiers, with each category representing a particular nucleotide. The count metric can include a count of each aptamer in the XNA aptamer library.

At block 315, the aptamers within the XNA aptamer library are partitioned into monoclonal compartments (e.g., monoclonal beads or compartmentalized droplets) for high throughput aptamer selection. For example, the aptamers may be attached to beads to generate a bead-based capture system for a target. Each bead may be attached to a unique aptamer sequence generating a library of monoclonal beads. The library of monoclonal beads may be generated by sequence-specific partitioning and covalent attachment of the sequences to the beads, which may be polystyrene, magnetic, glass beads, or the like. In some instances, the sequence-specific partitioning includes hybridization of XNA aptamers with capture oligonucleotides having an amine modified nucleotide for interaction with covalent attachment chemistries coated on the surface of a bead. In certain instances, the covalent attachment chemistries include N-hydroxysuccinimide (NHS) modified PEG, cyanuric chloride, isothiocyanate, nitrophenyl chloroformate, hydrazine, or any combination thereof. In some instances, UMIs are attached to the aptamers to enable accurate counting of copies of a given candidate sequence in elution or flow-through.

At block 320, a target (e.g., proteins, protein complexes, peptides, carbohydrates, inorganic molecules, cells, etc.) is obtained. The target may be obtained as a result of a query posed by a user (e.g., a client or customer). For example, a user may pose a query concerning identification of a hundred aptamers with the highest binding affinity for a given target or twenty aptamers with the greatest ability to inhibit activity of a given target. In some instances, the target is tagged with a label such as a fluorescent probe. At block 325, the bead-based capture system is incubated with the labeled target to allow for the aptamers to bind with the target and form aptamer-target complexes.

At block 330, the beads having aptamer-target complexes are separated from the beads having non-binding aptamers using a separation protocol. In some instances, the separation protocol includes a fluorescence-activated cell sorting system (FACS) to separate the beads having the aptamer-target complexes from the beads having non-binding aptamers. For example, a suspension of the bead-based capture system may be entrained in the center of a narrow, rapidly flowing stream of liquid. The flow may be arranged so that there is separation between beads relative to their diameter. A vibrating mechanism causes the stream of beads to break into individual droplets (e.g., one bead per droplet). Before the stream breaks into droplets, the flow passes through a fluorescence measuring station where the fluorescent label which is part of the aptamer-target complexes is measured. An electrical charging ring may be placed at a point where the stream breaks into droplets. A charge may be placed on the ring based on the prior fluorescence measurement, and the opposite charge is trapped on the droplet as it breaks from the stream. The charged droplets may then fall through an electrostatic deflection system that diverts droplets into containers based upon their charge (e.g., droplets having beads with aptamer-target complexes go into one container and droplets having beads with non-binding aptamers go into a different container). In some instances, the charge is applied directly to the stream, and the droplet breaking off retains a charge of the same sign as the stream. The stream may then returned to neutral after the droplet breaks off

At block 335, the aptamers from the aptamer-target complexes are eluted from the beads and target, and amplified by enzymatic or chemical processes to optionally prepare for subsequent rounds of selection (repeat blocks 310-330, for example a SELEX protocol). The stringency of the elution conditions can be increased to identify the tightest-binding or highest affinity sequences. In some instances, once the aptamers are separated and amplified, the aptamers may be sequenced to identify the sequence and optionally a count for each aptamer. Optionally, the separated non-binding aptamers are amplified by enzymatic or chemical processes. In some instances, once the non-binding aptamers are amplified, the non-binding aptamers may be sequenced to identify the sequence and optionally a count for each non-binding aptamer. The sequence and count of non-binding aptamers may provide information on which aptamers have the weakest binding (e.g., may be used in training of a machine-learning model), which may supplement or validate the results of the aptamers found to bind. If aptamers are high in count for non-binding and low in count for binding, then aptamers may be determined and validated to have a weak binding affinity. If certain aptamers have significant counts for both binding and non-binding, the aptamers may be limited for some other reason (e.g., competition for binding sites among same type of aptamers).

At block 340, a data set including the sequence, the count, and/or an analysis performed based on the separation protocol (e.g., a binary classifier or a multiclass classifier) for each aptamer that has gone through the selection process of steps 310-330 is processed for application in downstream machine-learning processes. The processing is performed by a controller/computer of platform 300. The data set may include the sequence, the count, and/or the analysis from the binding aptamers (those that formed the aptamer-target complexes), the non-binding aptamers (those that did not form the aptamer-target complexes), or the combination thereof. In general, there are different types of binders (e.g., agonist, antagonist, allosteric, etc.) and those would be characteristics that the system may be configured to distinguish between the different types of binders during training, testing, and/or experimental analysis. In some instances, the sequence, count, and/or analysis for each aptamer is processed for use as training data, test data, or validation data in one or more machine-learning models. In other instances, the sequence, count, and/or analysis for each aptamer is processed for use as actual experimental data in one or more trained machine-learning models. In either instance, the sequence, count, and/or analysis for each aptamer may be processed to generate selection sequence data comprising a representation of the sequence of each aptamer, a count metric, an analysis metric, or any combination thereof. The representation of the sequence can include one-hot encoding of each nucleotide in the sequence that maintains information about the order of the nucleotides in the aptamer. The representation of the sequence can additionally or alternatively include other features concerning the sequence and/or aptamer, for example, post-translational modifications, binding sites, enzyme active sites, local secondary structure, kmers or characteristics identified for specific kmers, etc. The representation of the sequence can additionally or alternatively include a string of category identifiers, with each category representing a particular nucleotide. The count metric may include a count of the aptamer detected subsequent to an exposure to the target (e.g., during incubation and potentially in the presence of other aptamers). In some instances, the count metric includes a count of the aptamer detected subsequent to an exposure to the target in each round of selection. The analysis metric may include a binary classifier such as functionally inhibited the target, functionally did not inhibit the target, bound to the target, or did not bound to the target, a fitness score, which is a measure of how well a given aptamer sequence performs as a solution with respect to the given problem, and/or a multiclass classifier such as a level of functional inhibition or a gradient scale for binding affinity.

In some instances, the processing in block 340 further includes (i) measuring the amount of noise in the data set, and (ii) provisioning components to dynamically change the architecture of the platform 300 for optimally addressing the measured amount of noise and label quality of the input sequences. As discussed herein, the less noise in the data set the more confidence there is to provision and configure components of the platform 300 to go from identifying or designing sequences in-sample domain (stay near training data) to out-of-sample domain (further away from training data). In certain instances, the amount of noise is expressed as a signal to noise ratio. The signal to noise ratio is used to measure the level of signal to the level of noise, and a larger signal to noise ratio means a higher signal quality. The signal and noise values for the ratio may be quantified using various techniques including measurements based on differences between the XNA aptamer library from block 310 and the data set obtained from block 335, or differences between the data set obtained from block 335 and inferred sets of sequences obtained from blocks 345(a)-345(n) (e.g., how far away the various sets of sequences are from one another and the greater the distance the greater the chance of noise). The controller/computer is able to select and optimize algorithms and models based on the determined signal to noise ratio (and implicitly the diversity of the sequences). For example, the controller/computer may modify the algorithms or models being used in blocks 345a-n, modify one or more model parameters, modify one or more hyperparameters, augment the input sequences or training set of data, modify the training/testing/validating approach, modify the objective or loss function for a given algorithm, or any combination thereof.

At blocks 345a-n, one or more machine-learning algorithms are trained by the controller/computer using the initial sequence data (from block 310), the selection sequence data (from block 335), or a combination thereof processed in block 340 to generate one or more trained machine-learning models. The one or more machine-learning models may include supervised models such as regression models (e.g., linear, decision tree, random forest, neural networks, etc.) or classification models (e.g., logistic regression, support vector machine, decision tree, random forest, neural networks, etc.) or unsupervised models such as clustering models (e.g., k-means, density-based, mean shift, etc.) or dimensionality reduction models (e.g., principle component analysis, etc.). In some instances (e.g., 345(a)), the machine-learning models include a neural network such as a feedforward neural network, a recurrent neural network, a convolutional neural network, or an ensemble of neural networks. In other instances, (e.g., 345(b)), the machine learning models include a linear model such as a regression model or a regularized regression model. The machine-learning algorithms may be trained using training data, test data, and validation data based on sets of initial sequence data and selection sequence data to predict fitness scores and identify aptamer sequences (e.g., aptamers not experimentally determined by a selection process but identified based on aptamers experimentally determined by a selection process) and optional counts and/or analytics for the identified aptamer sequences. An objective function or loss function, such as a Mean Square Error (MSE), likelihood loss, or log loss (cross entropy loss), may be used to train each of the one or more machine-learning models. In some instances, a machine-learning algorithm may be trained for predicting fitness scores and identifying aptamer sequences using the initial sequence data and/or the selection sequence data. Another machine-learning algorithm may be trained for predicting binding counts for the identified aptamer sequences using the initial sequence data and/or the selection sequence data. Another machine-learning algorithm may be trained for predicting analytics such as binding affinity for the identified aptamer sequences using the initial sequence data and/or the selection sequence data.

The trained machine-learning models are then be used to predict fitness scores and identify aptamer sequences and optional counts and/or analytics for the identified aptamer sequences. For example, a subset of the aptamers experimentally determined by the selection process to satisfy the query (e.g., aptamers that have high binding affinity with a target or predicted counts due primarily to high binding affinity with a target) can be identified and separated from aptamers experimentally determined by the selection process to not satisfy the query. The sequences for the subset of aptamers experimentally determined by the selection process to satisfy the query, sequences from a pool of sequences (e.g., a random pool of sequences or sequences pooled from a related librabry of sequences) different from the sequences from the subset of aptamers experimentally determined by the selection process, or a combination thereof can then be input into one or more machine learning models to predict fitness scores and identify in silico derived aptamer sequences (e.g., aptamer sequences that are derivatives of the experimentally selected aptamers) and optionally counts and analytics for the derived aptamer sequences. Optionally, the subset of the aptamers experimentally determined by the selection process that do not satisfy the query can also be input into one or more machine learning models to assist in identifying in silico derived aptamer sequences (e.g., aptamer sequences that are derivatives of the experimentally selected aptamers) and optionally counts and analytics for the derived aptamer sequences.

In some instances, additional techniques including the application of one or more different types of algorithms such as search algorithms (e.g., a genetic algorithm) or optimization algorithms (e.g., linear optimization) are used in combination with the one or more machine-learning models to improve upon the identification or design of aptamer sequences. For example, a subset of the aptamers experimentally determined by the selection process to satisfy the query can be identified and separated from aptamers experimentally determined by the selection process to not satisfy the query. This subset of aptamers, sequences from a pool of sequences different from the sequences from the subset of aptamers experimentally determined by the selection process, or a combination thereof may be used in a genetic search process that implements the trained machine-learning models as a learned fitness function for a genetic algorithm. The subset of aptamers can be input into the trained machine-learning models, which are used to predict fitness scores and identify in silico aptamer sequences for mating. Additionally, the trained machine-learning models (e.g., an ensemble off neural networks) may be configured to provide an uncertainty score regarding the predicted fitness score of a aptamer sequence as a binder, and the uncertainty score can be used in the genetic search process as at least part of a fitness score or as a filter for each identified aptamer sequence. The uncertainty score is determined using an uncertainty quantification process (e.g., a Gaussian process, a Monte Carlo dropout, non-Bayesian type processes, and the like) that quantifies uncertainty for predictions of the trained machine-learning models.

In the genetic algorithm, the subset of sequences experimentally determined by the selection process to satisfy the query, sequences from a pool of sequences different from the sequences from the subset of aptamers experimentally determined by the selection process, or a combination thereof serve as the initial population and a fitness function (i.e., the trained machine-learning model(s)) is used to determines how fit each aptamer sequence is (e.g., the ability of each sequence to compete as a binder with other sequences). The fitness function estimates or predicts a fitness score for each sequence. The probability that each sequence will be selected for reproduction is based on its fitness score and optionally may take into consideration the uncertainty score generated by trained machine-learning models for each predicted fitness score. Thereafter, pairs of sequences are selected based on their fitness scores. Sequences with high fitness have more chance to be selected for reproduction. Offspring are created by exchanging the genes (e.g., nucleotides) of parent sequences among themselves until a crossover point is reached. The new offspring are added to the population, and the process may be repeated until the population has converged (does not produce offspring which are significantly different from the previous generation). Then it may be determined that the genetic algorithm has identified or designed a set of solutions or sequences for binding to the given target. In certain instances, certain new offspring formed can be subjected to a mutation with a low random probability. This means that some of the nucleotides in the sequence can be randomly changed. In some instances, the genetic algorithm is constrained to control the cross over point and/or the mutations to a limited number of edits away from the training dataset.

At block 350, the output of the trained machine-learning models (identified aptamer sequences, fitness scores, and optional counts and/or analytics of the identified aptamer sequences) may trigger recording of some or all of the in silico identified aptamer sequences (e.g., positive and negative aptamer data such as predicted counts demonstrating increased binding affinity for a target or predicted counts demonstrating decreased binding affinity for a target) within a data structure (e.g., a database table). In some instances, the identified aptamer sequences are recorded in a data structure in association with additional information including the query (i.e., the given problem), the one or more targets that are the focus of the query and basis for the identification of the aptamer sequences, counts predicted for the aptamer sequences, fitness scores, analysis predicted for the aptamer sequences, or any combination thereof.

Additionally or alternatively, the output of the trained machine-learning models may trigger subsequent binding selections at blocks 310-335, or experimental testing or validation at block 355 to confirm the derived aptamers as strong therapeutic candidates that can bind the given molecular target. The actions executed in block 350 are dictated by the pipeline being executed by the aptamer development platform 300 for strategically identifying and generating high affinity binders of molecular targets. For example, in accordance with pipeline 100 illustrated in FIG. 1, the aptamer development platform 300 may perform: (i) a first round of binding selections at blocks 305-335, (ii) processing and input of derived aptamers into a first trained machine-learning model (e.g., an ensemble of neural networks) at blocks 340 and 345(a), (iii) a second round of binding selections at blocks 310-335, (iv) processing and input of derived aptamers into a second trained machine-learning model (e.g., a regression model) at blocks 340 and 345(b), and (v) experimental testing or validation at block 355 to confirm the derived aptamers as strong therapeutic candidates that can bind the given molecular target. Further, the actions executed in block 350 may be dictated dynamically by one or more factors including: the signal to noise ratio, the fitness score of the aptamer sequences, the uncertainty score of the aptamer sequences, predicted counts demonstrating increased binding affinity for a target, predicted counts demonstrating decreased binding affinity for a target, an absolute count of the aptamer sequences, or any combination thereof. For example, if the signal to noise ratio has achieved a predetermined threshold then subsequent binding selections and machine-learning identification or design may be avoided and the process may proceed to experimental testing or validation at block 355.

At block 355, experimental testing or validation is performed on some or all of the in silico aptamer sequences to experimentally measure analytics such as binding affinities with the target and/or binding affinities with one or more other targets. The experimental testing may be conditioned on input from a user. For example, a user device may present an interface in which the in silico aptamer sequences are identified along with input components configured to receive input to modify the in silico aptamer sequences (e.g., by removing or adding aptamers) and/or to generate an experiment-instruction communication to be sent to another device and/or other system. The experiment can include producing each of the in silico aptamer sequences. These aptamers can then be validated in the wet lab in either individual or bulk experiments using low throughput or high throughput assays. For example, the user can access a single aptamer (e.g. oligonucleotide). The single aptamer can be provided by an aptamer source, such as Twist Biosciences, Agilent, IDT, etc. The aptamer can be used to conduct biochemical assays (e.g. gel shift, surface plasma resonance, bio-layer interferometry, etc.). In some instances, multiple aptamers in a singular pool can be used to rerun the equivalent SELEX protocol (e.g., blocks 310-335) to identify enriched aptamers. Results can be assessed to determine whether the computational experiments are verified. In some instances, selections can be run in a digital format (i.e., ones that give a functional output per sequence) to validate particular sequences. In some instances, the validated sequences can be used to update the training set because the pair of sequence and affinity metric can be both normalized and calibrated.

As should be understood, the aptamer development platform 300 described with respect to FIG. 3 could be used for aptamer discovery where steps 310-335 are run in parallel to generate multiple monoclonal beads against multiple targets in association with one or more queries. Additionally or alternatively, the aptamer development platform 300 described with respect to FIG. 3 could be used for aptamer discovery where steps 310-335 are run in parallel to generate multiple monoclonal beads against multiple targets in association with one or more queries and identify in parallel aptamer sequences and optional counts and/or analytics for the identified aptamer sequences. The machine-learning models trained and used to make the predictions may be updated with results from the experiments and other machine-learning models using a distributed or collaborative learning approach such as federate learning which trains machine-learning models using decentralized data residing on end devices or systems. For example, a central or primary model may be updated or trained with results from all experiments being run and the results of the updating/training of the central or primary model may be propagated through to deployed secondary models (e.g., if information is obtained on cytokine a then the system may use that information to potential refine processes to identify for cytokine b).

### IV. MODELING PROCESSES AND TECHNIQUES TO IDENTIFY OR DESIGN SEQUENCES FOR BINDERS

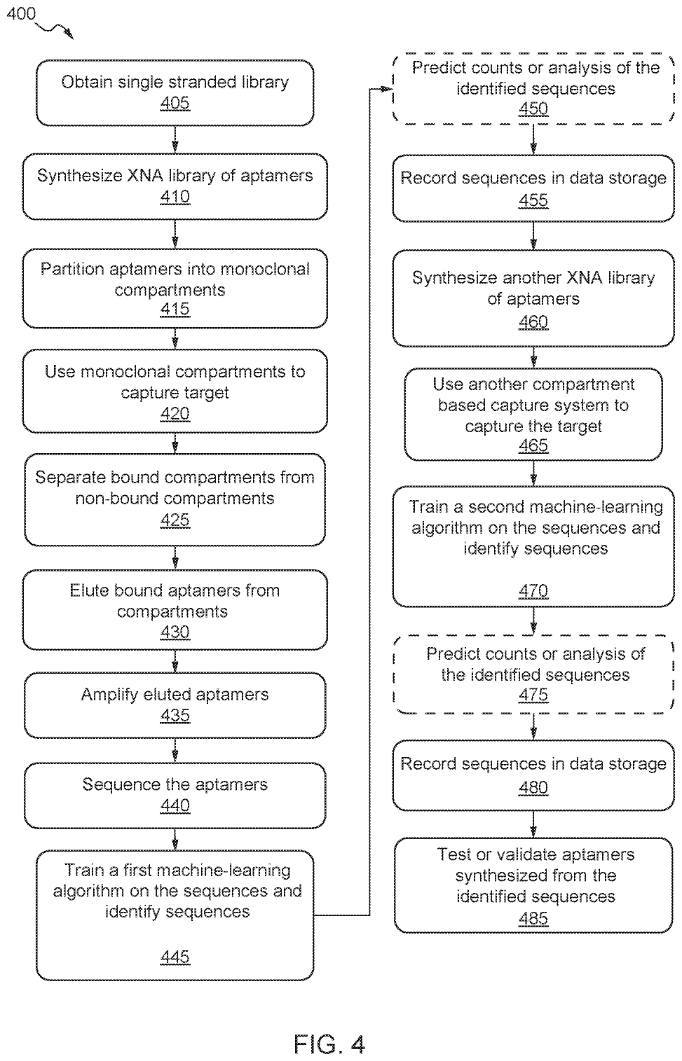

FIG. 4 is a simplified flowchart 400 illustrating an example of processing for developing aptamers using a machine-learning modeling system and an aptamer development platform (e.g., machine-learning modeling system 200 and the aptamer development platform 300 described with respect to FIGS. 2 and 3). Process 400 begins at block 405, at which one or more single stranded DNA or RNA (ssDNA or ssRNA) libraries are obtained. The one or more ssDNA or ssRNA libraries comprise a plurality of ssDNA or ssRNA sequences. At block 410, an XNA aptamer library is synthesized from the one or more ssDNA or ssRNA libraries. The XNA aptamer sequences that make up the XNA aptamer library may be synthesized in vitro with a transcription assay that includes enzymatic or chemical synthesis. The XNA aptamer library comprises a plurality of aptamer sequences. It will be appreciated that techniques disclosed herein can be applied to assess other aptamers rather than XNA aptamers. For example, alternatively or additionally, the techniques described herein may be used to assess the interactions between any type of sequence of nucleic acids (e.g., DNA and RNA) and epitopes of a target. Thus, the following block may synthesize a DNA or RNA aptamer library as input for aptamer sequences rather than constructing an XNA library.

At block 415, the plurality of aptamers within the XNA aptamer library (optionally DNA or RNA libraries) are partitioned into monoclonal compartments that combined establish a compartment-based capture system. Each monoclonal compartment comprises a unique aptamer from the plurality of aptamers. In some instances, the one or more monoclonal compartments are one or more monoclonal beads. In some instances, each monoclonal compartment or unique aptamer comprises a unique barcode (e.g., a unique molecular identifiers such as a unique sequence of nucleotides) for tracking identification of the compartment and/or the aptamer associated with the monoclonal compartment. At block 420, the compartment-based capture system is used to capture one or more targets. The capturing comprises the one or more targets binding to the unique aptamer within one or more monoclonal compartments. In some instances, the one or more targets are identified based on a query received from a user. As used herein, when an action is “based on” something, this means the action is based at least in part on at least a part of the something. At block 425, the one or more monoclonal compartments of the compartment-based capture system that comprise the one or more targets bound to the unique aptamer are separated from a remainder of monoclonal compartments of the compartment-based capture system that do not comprise the one or more targets bound to a unique aptamer. In some instances, the one or more monoclonal compartments are separated from the remainder of monoclonal compartments using a fluorescence-activated cell sorting system.

At block 430, the unique aptamer is eluted from each of the one or more monoclonal compartments and/or the one or more targets. At block 435, the unique aptamer from each of the one or more monoclonal compartments is amplified by enzymatic or chemical processes. At block 440, the unique aptamer from each of the one or more monoclonal compartments (e.g., the bound aptamers) are sequenced. The sequencing comprises using a sequencer to generate sequencing data and optionally analysis data for the unique aptamer from each of the one or more monoclonal compartments. The analysis data for the unique aptamer from each of the one or more monoclonal compartments may indicate the unique aptamer did bind to the one or more targets. In some instances, the sequencing further comprises generating count data for the unique aptamer from each of the one or more monoclonal compartments. In some instances, the sequencing further comprises sequences of unique aptamers from the remainder of the monoclonal compartments (e.g., non-bound aptamers). The sequencing further comprises using a sequencer to generate sequencing data and optionally analyze data for the unique aptamer from each of the remainder of the monoclonal compartments.

At block 445, the selection sequence data (from block 440) and optionally the count and analysis data are used for training a first machine-learning algorithm (e.g., a highly parametric machine-learning algorithm such as a neural network or ensemble of neural networks) to generate a first trained machine-learning model. Thereafter, aptamer sequences are identified, by the first trained machine-learning model, as an initial solution for a given problem. The identification may comprise inputting a subset of sequences from the selection sequence data (from block 440), sequences from a pool of sequences different from the sequences from the selection sequence data, or a combination thereof into the first trained machine-learning model, estimating, by the first trained machine-learning model, a fitness score of each input sequence (the fitness scores is a measure of how well a given sequence performs as a solution with respect to the given problem), and identifying aptamer sequences that satisfy the given problem based on the estimated fitness score for each sequence. In some instances, additional techniques including the application of one or more different types of algorithms such as search algorithms (e.g., a genetic algorithm) or optimization algorithms (e.g., linear optimization) are used in combination with the first trained machine-learning model to improve upon the identification of aptamer sequences. For example, the aptamer sequences identified by the first trained machine-learning model may be evolved using a genetic algorithm to identify or design aptamer sequences that satisfy the given problem, as described in detail herein.

Optionally at block 450, a count or analysis of the identified aptamer sequences is predicted by one or more prediction models. At block 455, the identified aptamer sequences and optionally the predicted analysis data and/or count data are recorded in a data structure in association with the one or more targets.

At block 460, another XNA aptamer library (optionally a DNA or RNA library) is synthesized from the identified aptamer sequences. The aptamers within the another XNA aptamer library (optionally a DNA or RNA library) are partitioned into monoclonal compartments that combined establish another compartment-based capture system. Each monoclonal compartment comprises a unique aptamer from the plurality of aptamers. At block 465, another compartment-based capture system is used to capture the one or more targets. The capturing comprises the one or more targets binding to the unique aptamer sequence within one or more monoclonal compartments. Thereafter, as described similarly with respect to blocks 425-440, the one or more monoclonal compartments of the another compartment-based capture system that comprise the one or more targets bound to the unique aptamer are separated from a remainder of monoclonal compartments of the another compartment-based capture system that does not comprise the one or more targets bound to a unique aptamer. The unique aptamer is then eluted from each of the one or more monoclonal compartments and/or the one or more targets, amplified by enzymatic or chemical processes, and sequenced.

At block 470, some or all of the selection sequence data (from block 440), the selection sequence data (from block 465), or a combination thereof are used for training a second machine-learning algorithm (e.g., a linear machine-learning algorithm such as a regression algorithm) to generate a second trained machine-learning model. Thereafter, aptamer sequences are identified, by the second trained machine-learning model, as a final solution for a given problem. The identification may comprise inputting a subset of sequences from the selection sequence data (from block 440), a subset of sequences from the selection sequence data (from block 465), sequences from a pool of sequences different from the sequences from the selection sequence data, or a combination thereof into the second trained machine-learning model, estimating, by the second trained machine-learning model, a fitness score of each input sequence (the fitness scores is a measure of how well a given sequence performs as a solution with respect to the given problem), and identifying aptamer sequences that satisfy the given problem based on the estimated fitness score for each sequence. In some instances, additional techniques including the application of one or more different types of algorithms such as search algorithms (e.g., a genetic algorithm) or optimization algorithms (e.g., linear optimization) are used in combination with the second trained machine-learning model to improve upon the identification or design of sequences for derived aptamers. For example, identification, by the second trained machine-learning model, of the aptamer sequences may be optimized using an optimization algorithm to identify or design aptamer sequences that satisfy the given problem, as described in detail herein.

Optionally at block 475, a count or analysis of the identified aptamer sequences is predicted by one or more prediction models. At block 480, the identified aptamer sequences and optionally the predicted analysis data and/or count data are recorded in a data structure in association with the one or more targets.

At block 485, the aptamer sequences identified as the final solution for the given problem are used to synthesize aptamers, which are then tested or validated as an aptamer capable of binding the target and solving the given problem.

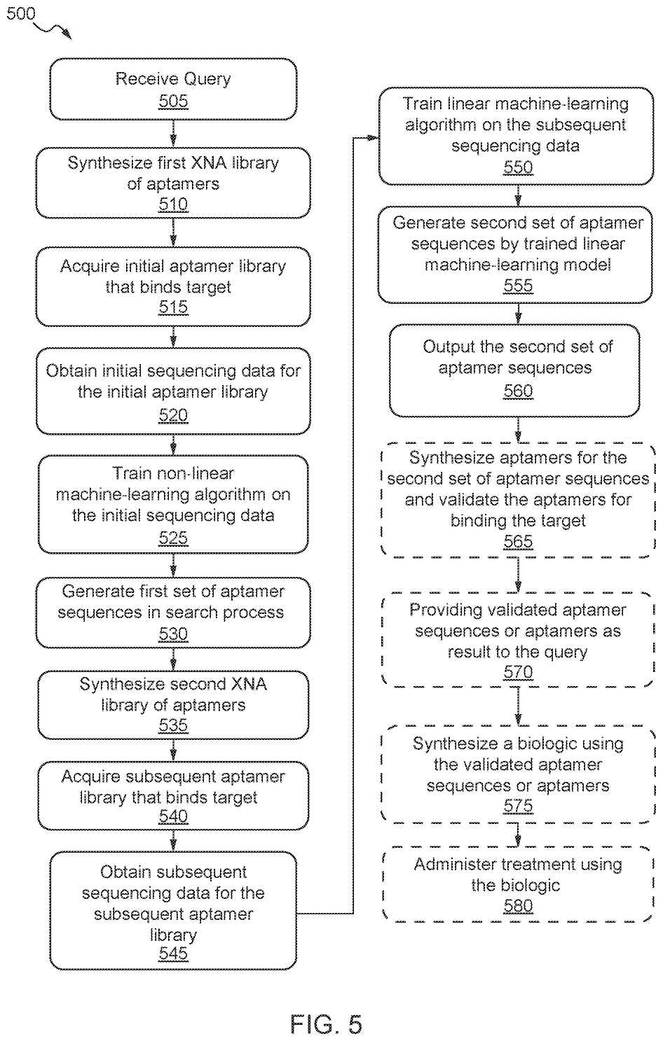

FIG. 5 is a simplified flowchart 500 illustrating an example of processing for developing aptamers using a predefined pipeline, machine-learning modeling system, and an aptamer development platform (e.g., pipeline 100, machine-learning modeling system 200 and the aptamer development platform 300 described with respect to FIGS. 1-3). Process 500 begins at block 505, at which a query is received concerning potential therapeutic candidates that can bind a target. For example, a user may pose a query concerning identification of a hundred aptamers with the highest binding affinity for a given target or a hundred aptamers with the greatest ability to inhibit activity of a given target. At block 510, a first XNA aptamer library is synthesized from one or more single stranded DNA or RNA (ssDNA or ssRNA) libraries, as described in detail with respect to flowchart 400 depicted in FIG. 4. At block 515, an initial aptamer library is acquired that potentially satisfies the query using a binding selection process (e.g., SELEX), as described in detail with respect to flowchart 400 depicted in FIG. 4. The intimal aptamer library comprises aptamers that bind to the target. At block 520, initial sequence data is obtained for each unique aptamer of the initial aptamer library that binds to the target. The sequencing comprises using a sequencer to generate sequencing data and optionally analysis data for the unique aptamer from each of the one or more monoclonal compartments, as described in detail with respect to flowchart 400 depicted in FIG. 4. The initial sequence data has a first signal to noise ratio. The first signal to noise ratio may be measured by: (i) quantifying a number of unique aptamers in block 515, quantifying a number of copies of each unique aptamer in block 515, and determining the sequencing depth of the sequencing data for each unique aptamer in block 520 (sequencing depth (also known as read depth) describes the number of times that a given nucleotide in an aptamer has been read in an experiment), and (ii) quantifying the first signal to noise ratio based on the quantification of the number of unique aptamers, the quantification of the copies of each unique aptamer, and the sequencing depth of the sequencing data for each unique aptamer.

At block 525, a nonlinear machine-learning algorithm is trained using a first set of training data comprising a subset of sequences from the initial sequence data (e.g., a training split that may only be 80% of the sequence data from block 520). The training includes iterative operations to find a set of parameters for nonlinear machine-learning algorithm that maximizes or minimizes an objective function (e.g., regression or classification loss) for the nonlinear machine-learning algorithm. Each iteration can involve finding a set of parameters for the algorithm so that the value of the objective function using the set of parameters is smaller than the value of the objective function using another set of parameters in a previous iteration. The objective function can be constructed to measure the difference between the outputs predicted using the nonlinear machine-learning algorithm and optional labels contained in the first set of training data. Once the set of parameters are identified, the nonlinear machine-learning algorithm has been trained and can be tested, validated, and/or utilized as a nonlinear machine-learning model for identification of aptamer sequences as designed. In certain instances, the nonlinear machine-learning model comprises greater than or equal to 10,000, 30,000, 50,000, or 75,000 parameters learned using: (i) the first set of training data comprising a subset of sequences from the initial sequence data, and (ii) a first objective function. In certain instances, the nonlinear machine-learning model comprises a neural network or an ensemble of neural networks.

At block 530, a first set of aptamer sequences is generated as an initial solution for a given problem using a search process. The first set of aptamer sequences is derived from the initial sequence data. Derived meaning that a model trained on the initial sequence data is used to identify completely new (de novo) sequences or evolve sequences from the initial sequence data. In some instances, the search process comprises (a) obtaining an initial population of aptamer sequences. The initial population is a subset of sequences from the initial sequence data (e.g., a production split that may only be 20% of the sequence data), sequences from a pool of sequences different from the sequences from the initial sequence data (e.g., a pool of entirely random sequences), or a combination thereof. The search process further comprises: (b) inputting the initial population into a nonlinear machine-learning model; (c) estimating, by the nonlinear machine-learning model, a fitness score of each aptamer sequence of the initial population, where the fitness scores is a measure of how well a given aptamer sequence performs as a solution with respect to the given problem; (d) selecting pairs of aptamer sequences from the initial population based on the fitness score for each aptamer sequence; (e) mating each pair of aptamer sequences by exchanging nucleotides between the pair of aptamer sequences up to a crossover point to generate offspring; (f) adding the offspring from each pair of aptamer sequences into a new population; (g) repeating steps (b)-(f) to create a sequence of new populations until a stopping criteria is met; and in response to meeting the stopping criteria, outputting a latest new population from step (f) as the first set of aptamer sequences.

In some instances, the estimating the fitness score of each aptamer sequence of the initial population, comprises generating, by the nonlinear machine-learning model, an uncertainty score for the fitness score of each aptamer sequence of the initial population. The uncertainty score is a quantification of uncertainty in a estimation of a fitness score by the nonlinear machine-learning model. The uncertainty score may be used: (1) at step (c) with the fitness function to calculate the fitness score and guide which steps the search algorithm takes through the fitness landscape, and/or (2) at step (d), (e), and/or (f) as a filter for which aptamers are selected to proceed to block 535. In certain instances, pairs of aptamer sequences from the initial population are selected based on the fitness score and uncertainty score for each aptamer sequence. Step (f) may further comprise adding some of the sequences that were mated to the new population based on the fitness score for each aptamer sequence. Step (e) may further comprise mutating one or more of the offspring or the sequences that were mated. Mutating comprises randomly changing one or more of the nucleotides in the offspring or the sequences that were mated. In some instances, the genetic algorithm is constrained to control the cross over point and/or the mutations to a limited number of edits away from the initial sequence data. The stopping criteria in step (g) may be (i) the number of generations reaches a maximum number of generations, (ii) after the running time reaches a maximum amount of time, (iii) when a value of the fitness function for the best point in the current population is less than or equal to a fitness limit, (iv) when the average relative change in the fitness function value over a maximum number of generations is less than a function tolerance, (v) there is no improvement in the objective function for a given period of time, (vi) the average relative change in the fitness function value over a maximum number of generations is less than a function tolerance, or any combination thereof.

At block 535, a second XNA aptamer library is synthesized from the first set of aptamer sequences, as described in detail with respect to flowchart 400 depicted in FIG. 4. At block 540, a subsequent aptamer library is acquired that potentially satisfies the query using a binding selection process (e.g., SELEX), as described in detail with respect to flowchart 400 depicted in FIG. 4. The subsequent aptamer library comprises aptamers that bind to the target. At block 545, subsequent sequence data is obtained for each unique aptamer of the subsequent aptamer library that binds to the target. The sequencing comprises using a sequencer to generate sequencing data and optionally analysis data for the unique aptamer from each of the one or more monoclonal compartments, as described in detail with respect to flowchart 400 depicted in FIG. 4. The subsequent sequence data has a second signal to noise ratio. In certain instances, the second signal to noise ratio is greater than the first signal to noise ratio. The second signal to noise ratio may be measured by: (i) quantifying a number of unique aptamers in block 540, quantifying a number of copies of each unique aptamer in block 540, and determining the sequencing depth of the sequencing data for each unique aptamer in block 545 (sequencing depth (also known as read depth) describes the number of times that a given nucleotide in an aptamer has been read in an experiment), and (ii) quantifying the second signal to noise ratio based on the quantification of the number of unique aptamers, the quantification of the copies of each unique aptamer, and the sequencing depth of the sequencing data for each unique aptamer.

At block 550, a linear machine-learning algorithm is trained using a second set of training data comprising a subset of sequences from the subsequent sequence data. The training includes iterative operations to find a set of parameters for linear machine-learning algorithm that maximizes or minimizes an objective function (e.g., regression or classification loss) for the linear machine-learning algorithm. Each iteration can involve finding a set of parameters for the algorithm so that the value of the objective function using the set of parameters is smaller than the value of the objective function using another set of parameters in a previous iteration. The objective function can be constructed to measure the difference between the outputs predicted using the linear machine-learning algorithm and optional labels contained in the second set of training data. Once the set of parameters are identified, the linear machine-learning algorithm has been trained and can be tested, validated, and/or utilized as a nonlinear machine-learning model for identification of aptamer sequences as designed. In certain instances, the linear machine-learning model comprises less than 10,000, 30,000, 50,000, or 75,000 parameters learned using: (i) the second set of training data comprising a subset of sequences from the subsequent sequence data, and (ii) a second objective function.

At block 555, a second set of aptamer sequences is generated by the linear machine-learning model as a final solution for the given problem. The second set of aptamer sequences is derived from the subsequent sequence data. Derived meaning that a model trained on the subsequent sequence data is used to identify completely new (de novo) sequences or evolve sequences from the subsequent sequence data. In some instances, the generating, by the linear machine-learning model, the second set of aptamer sequences, comprises: performing, using the subsequent sequence data, a linear regression analysis to quantify a relationship between independent and dependent variables; determining a contribution of each independent to a value of a dependent value based on the relationship between the independent and the dependent variables; identifying the second set of aptamer sequences based on the contribution of each independent to the value of the dependent value (e.g., predicting a fitness score and identifying aptamer sequences that satisfy a given fitness threshold); and outputting the second set of aptamer sequences. The second objective function may be optimized, by linear programming, under linear equality and/or inequality constraint of a loss function. Additionally or alternatively, regularized regression may be applied to the second objective function by constraining at least one coefficient to zero.

At block 560, the second set of aptamer sequences is output. For example, the second set of aptamer sequences may be locally presented (e.g., displayed) or transmitted to another device. The second set of aptamer sequences may be output along with an identifier of the target. In some instances, the second set of aptamer sequences is output to an end user or storage device. In some instances, the second set of aptamer sequences is output to an end user or storage device as a result to the query. At optional block 565, a final set of aptamers is synthesized using the second set of aptamer sequences, and one or more aptamers from the final set of aptamers are validated as being capable of binding the target and solving the given problem (e.g., binding with a predetermined binding affinity. The validating may be performed using a high throughput affinity assay such as a binding selection assay (e.g., phage display) or a low-throughput affinity assay such as BLI. In some instances, the predetermined binding affinity is a high binding affinity defined as K_{d}, Kᵢ, or IC₅₀≤250 nM (ΔG_{bind}≤−9 kcal/mol), which is a result from stronger intermolecular forces between an aptamer and the target leading to a longer residence time at the binding site (higher “on” rate, lower “off” rate). At optional block 570, upon validating the one or more aptamers and in response to the query, aptamer sequences for the one or more aptamers may be provided as a result to the query. At optional block 575, a biologic is synthesized using the one or more aptamers validated as being capable of binding the target and solving the given problem. The biologic may be used as a new drug, a therapeutic tool, a drug delivery device, diagnosis of disease, bio-imaging, analytical reagent, hazard detection, food inspection, and the like. At optional block 580, a treatment is administered to a subject with the biologic.

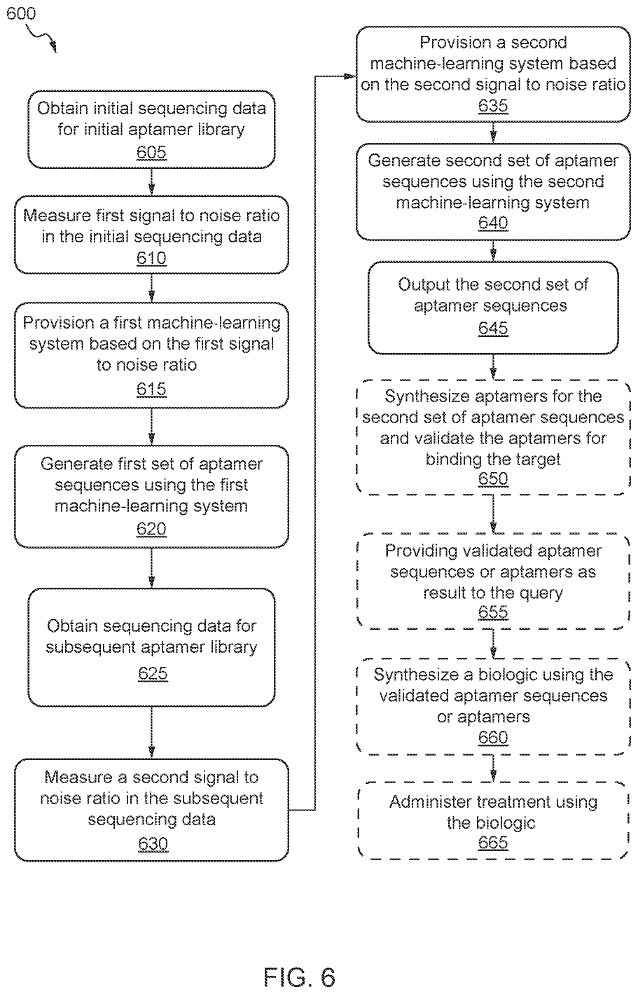

FIG. 6 is a simplified flowchart 600 illustrating an example of processing for developing aptamers using a dynamic pipeline, a machine-learning modeling system and an aptamer development platform (e.g., pipeline 100, machine-learning modeling system 200, and the aptamer development platform 300 described with respect to FIGS. 1-3). Process 600 begins at block 605, at which initial sequence data is obtained for each unique aptamer of an initial aptamer library that binds to the target. The initial sequence data may be obtained using a sequencer to generate sequencing data and optionally analysis data for the unique aptamer from each of the one or more monoclonal compartments, as described in detail with respect to flowchart 400 depicted in FIG. 4. The initial sequence data may be obtained in response receiving a query as described with respect to flowchart 500 depicted in FIG. 5. In some instances, the initial aptamer library is determined, using a binding selection process, from a first XNA aptamer library synthesized from one or more single stranded DNA or RNA libraries. At block 610, a first signal to noise ratio is measured within the initial sequence data. The first signal to noise ratio is measured by: (i) quantifying a number of unique aptamers, quantifying a number of copies of each unique aptamer, and determining the sequencing depth of the sequencing data for each unique aptamer (sequencing depth (also known as read depth) describes the number of times that a given nucleotide in an aptamer has been read in an experiment), and (ii) quantifying the first signal to noise ratio based on the quantification of the number of unique aptamers, the quantification of the copies of each unique aptamer, and the sequencing depth of the sequencing data for each unique aptamer.

At block 615, a first machine-learning system is provisioned, based on the first signal to noise ratio, for generating a first set of aptamer sequences derived from the initial sequence data. The provisioning comprises selecting or modifying one or more algorithms or models, modifying one or more model parameters of a preexisting algorithm or model, modifying one or more hyperparameters of a preexisting algorithm or model, augmenting the initial sequence data with additional data, selecting or modifying a training, testing, or validating approach for the one or more algorithms or the preexisting algorithm, modifying an objective or loss function of the one or more algorithms or the preexisting algorithm, or any combination thereof. In some instances, the one or more algorithms or models provisioned for the first machine-learning system comprise a first machine-learning model (e.g., a neural network model) and a search algorithm. The first machine-learning model may comprise model parameters learned using: (i) a first set of training data comprising a subset of sequences from the initial sequence data, and (ii) a first objective function, as described with respect to flowchart 500 depicted in FIG. 5. In such instances, the provisioning comprises selecting or modifying a first machine-learning algorithm or model and a search algorithm, modifying the model parameters of the first machine-learning algorithm or model, modifying one or more hyperparameters of the first machine-learning algorithm or model, augmenting the initial sequence data with additional data to generate the first set of training data, selecting or modifying a training, testing, or validating approach for the first machine-learning algorithm, modifying an objective or loss function of the first machine-learning algorithm, or any combination thereof.

At block 620, a first set of aptamer sequences is generated as an initial solution for a given problem using the first machine-learning system. The first set of aptamer sequences is derived from the initial sequence data. In some instances, the generating the first set of aptamer sequences comprises: inputting an initial population of aptamer sequences into the first machine-learning system; identifying, by applying the first machine-learning system, the first set of aptamer sequences; and outputting, by the first machine-learning system, the first set of aptamer sequences. In some instances, the initial population is a subset of sequences from the initial sequence data, sequences from a pool of sequences different from the sequences from the initial sequence data, or a combination thereof. In some instances, the first machine-learning system is applied by using a first machine-learning model as a fitness function in a search algorithm. The identifying may comprise predicting, by the first machine-learning model, a fitness score for each input sequence, and evolving, by the search algorithm, the input sequences into the first set of aptamer sequences based on the fitness score predicted for each input sequence.

In certain instances, the generating the first set of aptamer sequence comprises (a) obtaining an initial population of aptamer sequences. The initial population is a subset of sequences from the initial sequence data (e.g., a production split that may only be 20% of the sequence data), sequences from a pool of sequences different from the sequences from the initial sequence data (e.g., a pool of entirely random sequences), or a combination thereof. The generating further comprises: (b) inputting the initial population into a first machine-learning model; (c) estimating, by the first machine-learning model, a fitness score of each aptamer sequence of the initial population, where the fitness scores is a measure of how well a given aptamer sequence performs as a solution with respect to the given problem; (d) selecting pairs of aptamer sequences from the initial population based on the fitness score for each aptamer sequence; (e) mating each pair of aptamer sequences by exchanging nucleotides between the pair of aptamer sequences up to a crossover point to generate offspring; (f) adding the offspring from each pair of aptamer sequences into a new population; (g) repeating steps (b)-(f) to create a sequence of new populations until a stopping criteria is met; and in response to meeting the stopping criteria, outputting a latest new population from step (f) as the first set of aptamer sequences.

At block 625, subsequent sequence data is obtained for each unique aptamer of a subsequent aptamer library that binds to the target. The subsequent aptamer library comprises aptamers synthesized from the first set of aptamer sequences. The subsequent sequence data may be obtained using a sequencer to generate sequencing data and optionally analysis data for the unique aptamer from each of the one or more monoclonal compartments, as described in detail with respect to flowchart 400 depicted in FIG. 4. In some instances, the subsequent aptamer library is determined, using a binding selection process, from a second XNA aptamer library synthesized from the first set of aptamer sequences. At block 630, a second signal to noise ratio is measured within the subsequent sequence data. The second signal to noise ratio is measured by: (i) quantifying a number of unique aptamers, quantifying a number of copies of each unique aptamer, and determining the sequencing depth of the sequencing data for each unique aptamer (sequencing depth (also known as read depth) describes the number of times that a given nucleotide in an aptamer has been read in an experiment), and (ii) quantifying the second signal to noise ratio based on the quantification of the number of unique aptamers, the quantification of the copies of each unique aptamer, and the sequencing depth of the sequencing data for each unique aptamer.

At block 635, a second machine-learning system is provisioned, based on the second signal to noise ratio, for generating a second set of aptamer sequences derived from the subsequent sequence data. The provisioning comprises selecting or modifying one or more algorithms or models, modifying one or more model parameters of a preexisting algorithm or model, modifying one or more hyperparameters of a preexisting algorithm or model, augmenting the initial sequence data with additional data, selecting or modifying a training, testing, or validating approach for the one or more algorithms or the preexisting algorithm, modifying an objective or loss function of the one or more algorithms or the preexisting algorithm, or any combination thereof. In some instances, the one or more algorithms or models provisioned for the second machine-learning system comprise a second machine-learning model (e.g., a regression model). The second machine-learning model may comprise model parameters learned using: (i) a second set of training data comprising a subset of sequences from the subsequent sequence data, and (ii) a second objective function, as described with respect to flowchart 500 depicted in FIG. 5. In such instances, the provisioning comprises selecting or modifying a second machine-learning algorithm or model, modifying the model parameters of the second machine-learning algorithm or model, modifying one or more hyperparameters of the second machine-learning algorithm or model, augmenting the subsequent sequence data with additional data to generate the second set of training data, selecting or modifying a training, testing, or validating approach for the second machine-learning algorithm, modifying an objective or loss function of the second machine-learning algorithm, or any combination thereof.

At block 640, a second set of aptamer sequences is generated as a final solution for the given problem using the second machine-learning system. The second set of aptamer sequences is derived from the subsequent sequence data. In some instances, the generating, by the second machine-learning model, the second set of aptamer sequences, comprises: performing, by the second machine-learning model using the subsequent sequence data, a regression analysis to quantify a relationship between independent and dependent variables; determining, by the second machine-learning model, a contribution of each independent to a value of a dependent value based on the relationship between the independent and the dependent variables; identifying, by the second machine-learning model, the second set of aptamer sequences based on the contribution of each independent to the value of the dependent value; and outputting, by the second machine-learning model, the second set of aptamer sequences. The second objective function may be optimized, by linear programming, under linear equality and/or inequality constraint of a loss function. Additionally or alternatively, regularized regression may be applied to the second objective function by constraining at least one coefficient to zero. Additionally or alternatively, the second machine-learning system further comprises a search algorithm and the second machine-learning model and the search algorithm are used in conjunction to output the second set of aptamer sequences, as described with respect to the first machine-learning system.

At block 645, the second set of aptamer sequences is output. For example, the second set of aptamer sequences may be locally presented (e.g., displayed) or transmitted to another device. The second set of aptamer sequences may be output along with an identifier of the target. In some instances, the second set of aptamer sequences is output to an end user or storage device. In some instances, the second set of aptamer sequences is output to an end user or storage device as a result to the query. At optional block 650, a final set of aptamers is synthesized using the second set of aptamer sequences, and one or more aptamers from the final set of aptamers are validated as being capable of binding the target and solving the given problem (e.g., binding with a predetermined binding affinity). The validating may be performed using a high throughput affinity assay such as a binding selection assay (e.g., SELEX) or a low-throughput affinity assay such as BLI. In some instances, the predetermined binding affinity is a high binding affinity defined as K_{d}, Kᵢ, or IC₅₀≤250 nM (ΔG_{bind}≤−9 kcal/mol), which is a result from stronger intermolecular forces between an aptamer and the target leading to a longer residence time at the binding site (higher “on” rate, lower “off” rate). At optional block 655, upon validating the one or more aptamers and in response to the query, aptamer sequences for the one or more aptamers may be provided as a result to the query. At optional block 660, a biologic is synthesized using the one or more aptamers validated as being capable of binding the target and solving the given problem. The biologic may be used as a new drug, a therapeutic tool, a drug delivery device, diagnosis of disease, bio-imaging, analytical reagent, hazard detection, food inspection, and the like. At optional block 665, a treatment is administered to a subject with the biologic.

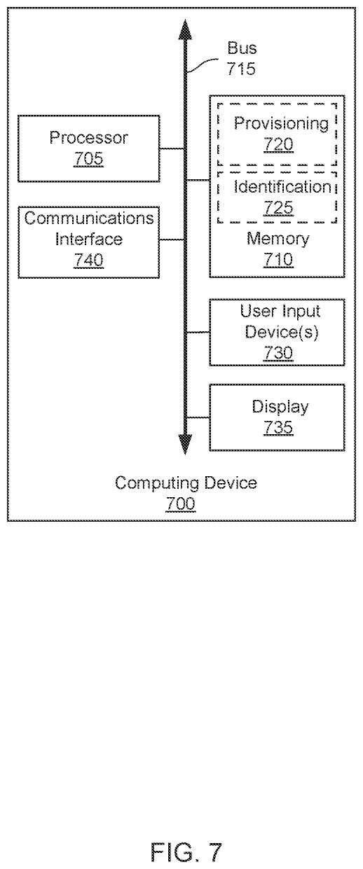

FIG. 7 illustrates an example computing device 700 suitable for use with systems and methods for developing aptamers and biologics or providing results to a query according to this disclosure. The example computing device 700 includes a processor 505 which is in communication with the memory 710 and other components of the computing device 700 using one or more communications buses 715. The processor 705 is configured to execute processor-executable instructions stored in the memory 710 to perform one or more methods for developing aptamers or biologics or providing results to a query according to different examples, such as part or all of the example method 400, 500, or 600 described above with respect to FIG. 4, 5, or 6. In this example, the memory 710 stores processor-executable instructions that provide for provisioning of machine-learning algorithms or models 720 and aptamer identification 725, as discussed above with respect to FIGS. 1-6 (e.g., the controller/computer of platform 300).

The computing device 700, in this example, also includes one or more user input devices 730, such as a keyboard, mouse, touchscreen, microphone, etc., to accept user input. The computing device 700 also includes a display 735 to provide visual output to a user such as a user interface or display of aptamer sequences. The computing device 700 also includes a communications interface 740. In some examples, the communications interface 740 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### V. ADDITIONAL CONSIDERATIONS

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method comprising:
obtaining initial sequence data for each unique aptamer of an initial aptamer library that binds to a target, wherein the initial sequence data has a first signal to noise ratio;
generating, by a search process, a first set of aptamer sequences as an initial solution for a given problem, wherein the first set of aptamer sequences are derived from the initial sequence data;
obtaining subsequent sequence data for each unique aptamer of a subsequent aptamer library that binds to the target, wherein the subsequent aptamer library comprises aptamers synthesized from the first set of aptamer sequences, and wherein the subsequent sequence data has a second signal to noise ratio that is greater than the first signal to noise ratio;
generating, by a linear machine-learning model, a second set of aptamer sequences as a final solution for the given problem, wherein the second set of aptamer sequences are derived from the subsequent sequence data; and
outputting the second set of aptamer sequences.

2. The method of claim 1, wherein the search process comprises:
(a) obtaining an initial population of aptamer sequences, wherein the initial population is a subset of sequences from the initial sequence data, sequences from a pool of sequences different from the sequences from the initial sequence data, or a combination thereof;
(b) inputting the initial population into a nonlinear machine-learning model;
(c) estimating, by the nonlinear machine-learning model, a fitness score of each aptamer sequence of the initial population, wherein the fitness scores is a measure of how well a given aptamer sequence performs as a solution with respect to the given problem;
(d) selecting pairs of aptamer sequences from the initial population based on the fitness score for each aptamer sequence;
(e) mating each pair of aptamer sequences by exchanging nucleotides between the pair of aptamer sequences up to a crossover point to generate offspring;
(f) adding the offspring from each pair of aptamer sequences into a new population;
(g) repeating steps (b)-(f) to create a sequence of new populations until a stopping criteria is met; and
in response to meeting the stopping criteria, outputting a latest new population from step (f) as the first set of aptamer sequences.

3. The method of claim 2, wherein:
the estimating the fitness score of each aptamer sequence of the initial population, comprises generating, by the nonlinear machine-learning model, an uncertainty score for the fitness score of each aptamer sequence of the initial population;
the uncertainty score is a quantification of uncertainty in a estimation of a fitness score by the nonlinear machine-learning model; and
pairs of aptamer sequences from the initial population are selected based on the fitness score and uncertainty score for each aptamer sequence.

4. The method of claim 2, wherein the generating, by the linear machine-learning model, the second set of aptamer sequences, comprises:
performing, using the subsequent sequence data, a linear regression analysis to quantify a relationship between independent and dependent variables;
determining a contribution of each independent to a value of a dependent value based on the relationship between the independent and the dependent variables;
identifying the second set of aptamer sequences based on the contribution of each independent to the value of the dependent value; and
outputting the second set of aptamer sequences.

5. The method of claim 4, wherein:
the nonlinear machine-learning model comprises greater than or equal to 10,000 parameters learned using: (i) a first set of training data comprising a subset of sequences from the initial sequence data, and (ii) a first objective function;
the linear machine-learning model comprises less than 10,000 parameters learned using: (i) a second set of training data comprising a subset of sequences from the subsequent sequence data, and (ii) a second objective function;
the second objective function is optimized, by linear programming, under linear equality and/or inequality constraint of a loss function; and
regularized regression is applied to the second objective function by constraining at least one coefficient to zero.

6. The method of claim 1, further comprising:
synthesizing a final set of aptamers using the second set of aptamer sequences;
validating, using a high-throughput or low-throughput affinity assay, one or more aptamers from the final set of aptamers capable of binding the target and solving the given problem; and
synthesizing a biologic using the one or more aptamers validated as being capable of binding the target and solving the given problem.

7. The method of claim 1, further comprising:
receiving a query concerning potential therapeutic candidates that can bind the target and solve the given problem;
acquiring the initial aptamer library as potentially satisfying the query;
synthesizing a final set of aptamers using the second set of aptamer sequences;
validating, using a high-throughput or low-throughput affinity assay, one or more aptamers from the final set of aptamers capable of binding the target and solving the given problem; and
upon validating the one or more aptamers and in response to the query, providing aptamer sequences for the one or more aptamers as a result to the query.

8. A computer-program product tangibly embodied in a non-transitory machine-readable storage medium, including instructions configured to cause one or more data processors to perform actions including:
obtaining initial sequence data for each unique aptamer of an initial aptamer library that binds to a target, wherein the initial sequence data has a first signal to noise ratio;
generating, by a search process, a first set of aptamer sequences as an initial solution for a given problem, wherein the first set of aptamer sequences are derived from the initial sequence data;
obtaining subsequent sequence data for each unique aptamer of a subsequent aptamer library that binds to the target, wherein the subsequent aptamer library comprises aptamers synthesized from the first set of aptamer sequences, and wherein the subsequent sequence data has a second signal to noise ratio that is greater than the first signal to noise ratio;
generating, by a linear machine-learning model, a second set of aptamer sequences as a final solution for the given problem, wherein the second set of aptamer sequences are derived from the subsequent sequence data; and
outputting the second set of aptamer sequences.

9. The computer-program product of claim 8, wherein the search process comprises:
(a) obtaining an initial population of aptamer sequences, wherein the initial population is a subset of sequences from the initial sequence data, sequences from a pool of sequences different from the sequences from the initial sequence data, or a combination thereof;
(b) inputting the initial population into a nonlinear machine-learning model;
(c) estimating, by the nonlinear machine-learning model, a fitness score of each aptamer sequence of the initial population, wherein the fitness scores is a measure of how well a given aptamer sequence performs as a solution with respect to the given problem;
(d) selecting pairs of aptamer sequences from the initial population based on the fitness score for each aptamer sequence;
(e) mating each pair of aptamer sequences by exchanging nucleotides between the pair of aptamer sequences up to a crossover point to generate offspring;
(f) adding the offspring from each pair of aptamer sequences into a new population;
(g) repeating steps (b)-(f) to create a sequence of new populations until a stopping criteria is met; and
in response to meeting the stopping criteria, outputting a latest new population from step (f) as the first set of aptamer sequences.

10. The computer-program product of claim 9, wherein:
the estimating the fitness score of each aptamer sequence of the initial population, comprises generating, by the nonlinear machine-learning model, an uncertainty score for the fitness score of each aptamer sequence of the initial population;
the uncertainty score is a quantification of uncertainty in a estimation of a fitness score by the nonlinear machine-learning model; and
pairs of aptamer sequences from the initial population are selected based on the fitness score and uncertainty score for each aptamer sequence.

11. The computer-program product of claim 9, wherein the generating, by the linear machine-learning model, the second set of aptamer sequences, comprises:
performing, using the subsequent sequence data, a linear regression analysis to quantify a relationship between independent and dependent variables;
determining a contribution of each independent to a value of a dependent value based on the relationship between the independent and the dependent variables;
identifying the second set of aptamer sequences based on the contribution of each independent to the value of the dependent value; and
outputting the second set of aptamer sequences.

12. The computer-program product of claim 11, wherein:
the nonlinear machine-learning model comprises greater than or equal to 10,000 parameters learned using: (i) a first set of training data comprising a subset of sequences from the initial sequence data, and (ii) a first objective function;
the linear machine-learning model comprises less than 10,000 parameters learned using: (i) a second set of training data comprising a subset of sequences from the subsequent sequence data, and (ii) a second objective function;
the second objective function is optimized, by linear programming, under linear equality and/or inequality constraint of a loss function; and
regularized regression is applied to the second objective function by constraining at least one coefficient to zero.

13. The computer-program product of claim 8, wherein the actions further comprise:
synthesizing a final set of aptamers using the second set of aptamer sequences;
validating, using a high-throughput or low-throughput affinity assay, one or more aptamers from the final set of aptamers capable of binding the target and solving the given problem; and
synthesizing a biologic using the one or more aptamers validated as being capable of binding the target and solving the given problem.

14. The computer-program product of claim 1, wherein the actions further comprise:
receiving a query concerning potential therapeutic candidates that can bind the target and solve the given problem;
acquiring the initial aptamer library as potentially satisfying the query;
synthesizing a final set of aptamers using the second set of aptamer sequences;
validating, using a high-throughput or low-throughput affinity assay, one or more aptamers from the final set of aptamers capable of binding the target and solving the given problem; and
upon validating the one or more aptamers and in response to the query, providing aptamer sequences for the one or more aptamers as a result to the query.

15. A system comprising:
one or more data processors; and
a non-transitory computer readable storage medium containing instructions which, when executed on the one or more data processors, cause the one or more data processors to perform actions including:
obtaining initial sequence data for each unique aptamer of an initial aptamer library that binds to a target, wherein the initial sequence data has a first signal to noise ratio;
generating, by a search process, a first set of aptamer sequences as an initial solution for a given problem, wherein the first set of aptamer sequences are derived from the initial sequence data;
obtaining subsequent sequence data for each unique aptamer of a subsequent aptamer library that binds to the target, wherein the subsequent aptamer library comprises aptamers synthesized from the first set of aptamer sequences, and wherein the subsequent sequence data has a second signal to noise ratio that is greater than the first signal to noise ratio;
generating, by a linear machine-learning model, a second set of aptamer sequences as a final solution for the given problem, wherein the second set of aptamer sequences are derived from the subsequent sequence data; and
outputting the second set of aptamer sequences.

16. The system of claim 15, wherein the search process comprises:
(a) obtaining an initial population of aptamer sequences, wherein the initial population is a subset of sequences from the initial sequence data, sequences from a pool of sequences different from the sequences from the initial sequence data, or a combination thereof;
(b) inputting the initial population into a nonlinear machine-learning model;
(c) estimating, by the nonlinear machine-learning model, a fitness score of each aptamer sequence of the initial population, wherein the fitness scores is a measure of how well a given aptamer sequence performs as a solution with respect to the given problem;
(d) selecting pairs of aptamer sequences from the initial population based on the fitness score for each aptamer sequence;
(e) mating each pair of aptamer sequences by exchanging nucleotides between the pair of aptamer sequences up to a crossover point to generate offspring;
(f) adding the offspring from each pair of aptamer sequences into a new population;
(g) repeating steps (b)-(f) to create a sequence of new populations until a stopping criteria is met; and
in response to meeting the stopping criteria, outputting a latest new population from step (f) as the first set of aptamer sequences.

17. The system of claim 15, wherein:
the estimating the fitness score of each aptamer sequence of the initial population, comprises generating, by the nonlinear machine-learning model, an uncertainty score for the fitness score of each aptamer sequence of the initial population;
the uncertainty score is a quantification of uncertainty in a estimation of a fitness score by the nonlinear machine-learning model; and
pairs of aptamer sequences from the initial population are selected based on the fitness score and uncertainty score for each aptamer sequence.

18. The system of claim 15, wherein the generating, by the linear machine-learning model, the second set of aptamer sequences, comprises:
performing, using the subsequent sequence data, a linear regression analysis to quantify a relationship between independent and dependent variables;
determining a contribution of each independent to a value of a dependent value based on the relationship between the independent and the dependent variables;
identifying the second set of aptamer sequences based on the contribution of each independent to the value of the dependent value; and
outputting the second set of aptamer sequences.

19. The system of claim 18, wherein:
the nonlinear machine-learning model comprises greater than or equal to 10,000 parameters learned using: (i) a first set of training data comprising a subset of sequences from the initial sequence data, and (ii) a first objective function;
the linear machine-learning model comprises less than 10,000 parameters learned using: (i) a second set of training data comprising a subset of sequences from the subsequent sequence data, and (ii) a second objective function;
the second objective function is optimized, by linear programming, under linear equality and/or inequality constraint of a loss function; and
regularized regression is applied to the second objective function by constraining at least one coefficient to zero.

20. The system of claim 15, wherein the actions further comprise:
receiving a query concerning potential therapeutic candidates that can bind the target and solve the given problem;
acquiring the initial aptamer library as potentially satisfying the query;
synthesizing a final set of aptamers using the second set of aptamer sequences;
validating, using a high-throughput or low-throughput affinity assay, one or more aptamers from the final set of aptamers capable of binding the target and solving the given problem; and
upon validating the one or more aptamers and in response to the query, providing aptamer sequences for the one or more aptamers as a result to the query.
